(19) United States
(12) Patent Application Publication
(10) Pub. No.: US 2024/0148994 A1
(43) Pub. Date: May 9, 2024

(54) METHODS AND SYSTEMS FOR TRANSFERRING ACCESS TO PATIENT DATA BETWEEN ORGANIZATIONS

(71) Applicant: ResMed Digital Health Inc., San Diego, CA (US)

(72) Inventors: Shweta PAI, Halifax (CA); Robert Daniel NADLER, San Diego, CA (US); Manish LADHA, Redmond, WA (US); Kiran THYAGARAJ, San Diego, CA (US); Norman Jay MACDONALD, Halifax (CA); Joseph Van Daniel MARTIN, Halifax (CA); Timothy HOFLER, Escondido, CA (US)

(73) Assignee: ResMed Digital Health Inc., San Diego, CA (US)

(21) Appl. No.: 18/500,776

(22) Filed: Nov. 2, 2023

Related U.S. Application Data
(60) Provisional application No. 63/422,173, filed on Nov. 3, 2022.

(51) Int. Cl. A61M 16/00; G16H 10/60

(52) U.S. Cl. CPC A61M 16/024; G16H 10/60; A61M 2205/3553; A61M 2205/3584; A61M 2205/502; A61M 2205/52; A61M 2205/60

(57) ABSTRACT

Systems and methods provide transferring of access to patient data between organizations. In some implementations, one or more servers, with various services (12, 14, 16, 18, 20), transfer access to patient data from a first to a second organization. The server(s) may generate a user interface; receive job input data via the interface; and generate a transfer job based on the job input data. The transfer job may include list data indicative of one or more patients. The server(s) may execute the generated transfer job. The executing iterates through the list data and communicates transfer commands to services of the server(s). Access to the patient data associated with the patient(s) of central database(s) indicated by the list data is transferred from the first organization to the second organization. The server(s) may generate, for a display on the user interface, transfer job status information concerning the status of the executing.

### CROSS-REFERENCE TO RELATED APPLICATIONS

This application claims the benefit of U.S. Provisional Application No. 63/422,173, filed 3 Nov. 2022, the entire disclosure of which is hereby incorporated herein by reference.

### FIELD OF THE TECHNOLOGY

The present technology relates to methods and systems for transferring access to patient data, and more particularly, to methods and systems for transferring access to patient data, such as the data associated with use and/or operation of a medical device, such as a respiratory pressure therapy device, from one organization to the other organization.

### DESCRIPTION OF RELATED ART

When an organization (e.g., a health management organization such as a Home Medical Equipment (HME) company) acquires or merges with another similar organization, the patient data, such as the electronic data associated with the use and/or operation of a home medical equipment, such as a respiratory therapy device, of the acquired organization needs to be merged or consolidated with the similar patient data of the acquiring organization for the internal systems to operate properly. In this regard, the new organization will typically need access to historic data of the use and/or operation of each of the users of such devices that typically generate/accumulate data on a daily basis for each user. Prior to such a transfer, such data of both organizations may be discretely maintained in the data systems (e.g., server(s), list(s) and/or database(s)) of a management entity. Typically, such patient data of the acquired organization is consolidated with the patient data of the acquiring organization by transferring or enabling access to the acquired organization's patient data for each of the patients managed by the acquired organization after receiving a transfer request, such as from an administrator of the acquired organization and/or the acquiring organization. This may also involve updating certain patient data associated with the rules and requisites of the receiving organization.

To accomplish such a transfer, a conventional method may involve a programmer creating and executing custom database scripts for the particular transfer to change the access for the multitude of constituents of the patient data for each of the patients from one organization to another organization. However, the conventional method is time intensive and not particularly effective as it tends to result in integrity issues within the patient data due to the complexity of the organization of the constituents of such data.

Accordingly, there is a need for an improved system and method for transferring patient access to patient data between organizations.

### SUMMARY OF THE TECHNOLOGY

Systems and methods provide for a transfer of access to patient data between organizations.

Some implementations of the present technology may include a method of one or more servers for transferring access to patient data from a first organization to a second organization. The patient data may include therapy data for one or more patients. The method may include generating, by the one or more servers, a user interface. The method may include receiving job input data via the user interface, the job input data may include transfer information indicating the first organization and the second organization. The method may include generating, by the one or more servers, a transfer job based on the job input data. The transfer job may include list data indicative of the one or more patients, the one or more patients being in association with the first organization. The method may include executing, by the one or more servers, the generated transfer job. The executing may include iterating through the list data and, for each patient indicated by the list data, communicating transfer commands to a plurality of data services of the one or more servers, whereby access to the patient data, of a central database, associated with the one or more patients indicated by the list data transfers from the first organization to the second organization. The method may include generating, for a display on the user interface, transfer job status information concerning the status of the executing.

In some implementations, the plurality of data services may include a billing rules system, a compliance rules system, a list service and a device type subset data system. The user interface may prompt for input of a target entity identification and source entity identification and generates a network communication to a merge coordinator service with the input target entity identification and input source entity identification. The merge coordinator service may receive the network communication with the entity identification and source entity identification and query the central database to generate a list of patients associated with the source entity identification. The user interface may further prompt for input of a clinical user or location of the source entity and generate the network communication to the merge coordinator service with the clinical user or location of the source entity. The user interface may further prompts for input of a clinical user or location of the target entity and generates the network communication to the merge coordinator service with the clinical user or location of the target entity. The user interface may further prompt for input of a clinical user or location of the target entity and generate the network communication to the merge coordinator service with the clinical user or location of the target entity. The merge coordinator service may iteratively communicate with a patient transfer service for each patient of the list of patients. The patient transfer service may communicate with the plurality of data services and the central database in response to each iterative communication from the merge coordinator service to transfer access to a patient of the list of patients from the first organization to the second organization. The user interface may present a restart icon in association with a failed transfer, and wherein the user interface may be configured to communicate a restart request for the failed transfer in response to activation of the restart icon. The merge coordinator service may communicate with a database to store the transfer job status information. The patient data for which access may be transferred by the executing may include therapy device data. The method may further include communicating, with one or more servers, one or more control commands to a patient therapy device based on the therapy device data for making a change to an operation of the therapy device.

Some versions of the present technology may include a system for transferring access to patient data from a first organization to a second organization. The patient data may include therapy data for one or more patients. The system may include one or more processors. The system may include one or more computer-readable storage media having program instructions thereon, which when executed by the one or more processors cause the system to generate a user interface. The instructions may cause the system to receive job input data via the user interface. The job input data may include transfer information indicating the first organization and the second organization. The instructions may cause the system to generate a transfer job based on the job input data, the transfer job may include list data indicative of the one or more patients. The one or more patients may be in association with the first organization. The instructions may cause the system to execute the generated transfer job. The execution may include iterating through the list data and, for each patient indicated by the list data, communicating transfer commands to a plurality of data services of the one or more servers, whereby access to the patient data, of a central database, associated with the one or more patients indicated by the list data transfers from the first organization to the second organization. The instructions may cause the system to generate, for a display on the user interface, transfer job status information concerning the status of the execution.

In some implementations, the plurality of data services may include a billing rules system, a compliance rules system, a list service and a device type subset data system. The user interface may be configured to prompt for input of a target entity identification and source entity identification and generates a network communication to a merge coordinator service with the input target entity identification and input source entity identification. The merge coordinator service may be configured to receive the network communication with the entity identification and source entity identification and may be further configured to query the central database to generate a list of patients associated with the source entity identification. The user interface may be configured to further prompt for input of a clinical user or location of the source entity and may be further configured to generate the network communication to the merge coordinator service with the clinical user or location of the source entity. The user interface may be configured to further prompt for input of a clinical user or location of the target entity and may be configured to generate the network communication to the merge coordinator service with the clinical user or location of the target entity. The user interface may be further configured to prompt for input of a clinical user or location of the target entity and may be configured to generate the network communication to the merge coordinator service with the clinical user or location of the target entity. The merge coordinator service may be configured to iteratively communicate with a patient transfer service for each patient of the list of patients. The patient transfer service may be configured to communicate with the plurality of data services and the central database in response to each iterative communication from the merge coordinator service to transfer access to a patient of the list of patients from the first organization to the second organization. The user interface may be configured to present a restart icon in association with a failed transfer, and wherein the user interface may be configured to communicate a restart request for the failed transfer in response to activation of the restart icon. The patient data for which access may be transferred by the execution of the generated transfer job may include therapy device data, and the system may be further figured to communicate, by one or more servers, one or more control commands to a patient therapy device based on the therapy device data for making a change to an operation of the therapy device.

Some implementations of the present technology may include a method of one or more servers for transferring access to patient data from a first organization to a second organization. The patient data may include therapy data for a patient using a therapy device. The method may include generating, by the one or more servers, a user interface for transferring access to the patient data. The method may include receiving input data from the second organization, with the user interface, the input data may include device identification indicia of the therapy device. The method may include, based on receiving the device identification indicia, communicating transfer commands to a plurality of services of the one or more servers, whereby access to the patient data for the patient of a central database transfers from the first organization to the second organization. The method may include generating, for a display on the user interface, an indication of the transfer of access.

In some implementations, the user interface prompts for input of serial number of the therapy device and activates a search of the central database with the serial number. In response to search, the user interface may display patient data for a patient associated with: (a) the first organization and (b) the device identification indicia of the therapy device. The user interface may prompt for entry of confirmation of authority to request transfer of the patient from the first organization to the second organization. The user interface may prompt for entry of a clinical user of the second organization and/or a location of the second location. The user interface may display the indication of the transfer of access to the second organization. The method may further include generating a further user interface and communicating the further user interface to the first organization, the further user interface may include a display of the indication of the transfer of access. The patient data, for which access may be transferred based on the communicated transfer commands, may include therapy device data associated with the therapy device, and the method may further include communicating, with one or more servers, one or more control commands to the therapy device based on the therapy device data for making a change to an operation of the therapy device.

Some implementations of the present technology may include a system configured to transfer access to patient data from a first organization to a second organization. The patient data may include therapy data for one or more patients. The system may include one or more processors. The system may include one or more computer-readable storage media having program instructions thereon, which when executed by the one or more processors cause the system to generate a user interface for transferring access to the patient data. The executed instructions may cause the system to receive input data from the second organization, with the user interface, the input data may include device identification indicia of the therapy device. The executed instructions may cause the system to based on the received device identification indicia, communicate transfer commands to a plurality of services of the one or more servers, whereby access to the patient data for the patient of a central database transfers from the first organization to the second organization. The executed instructions may cause the system to generate, for a display on the user interface, an indication of the transfer of access.

In some implementations, the user interface may be configured to prompt for input of serial number of the therapy device, and activate a search of the central database with the serial number. In response to the search, the user interface may be configured to display patient data for a patient associated with: (a) the first organization and (b) the device identification indicia of the therapy device. The user interface may be configured to prompt for entry of confirmation of authority to request transfer of the patient from the first organization to the second organization. The user interface may be configured to prompt for entry of a clinical user of the second organization and/or a location of the second location. The user interface may be configured to display the indication of the transfer of access to the second organization. The system may be further configured to generate a further user interface and communicate the further user interface to the first organization, wherein the further user interface may be configured to display the indication of the transfer of access. The patient data for which access may be transferred based on the communicated transfer commands, may include therapy device data associated with the therapy device, and wherein the method further may include communicating, with one or more servers, one or more control commands to the therapy device based on the therapy device data for making a change to an operation of the therapy device.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

The present technology is illustrated by way of example, and not by way of limitation, in the figures of the accompanying drawings, in which like reference numerals refer to similar elements including:

FIG. 1 is a system diagram illustrating a flow of communications involving a transfer tool for managing a transfer of data access within a patient data management system according to an example of the present technology.

FIGS. 2-11 illustrate various graphic displays for an example user interface that may be generated in relation to the operations of the transfer tool of FIG. 1.

FIG. 12 illustrates an example process of such a transfer tool in accordance with some implementations of the present technology.

FIG. 13 is another system diagram illustrating a flow of communications for managing a transfer of data access within a patient data management system according to another example of the present technology.

FIGS. 14-18 illustrate various graphic displays for an example user interface that may be generated in relation to the operations of the system of FIG. 13.

FIG. 19 illustrates an example process for data access transfer such as in accordance with the operations of the system of FIG. 13.

FIG. 20 illustrates a block diagram of an illustrative example of a computing system.

FIG. 21A shows an example system in accordance with the present technology. A patient 1000 wearing a patient interface 3000 receives a supply of pressurised air from an RPT device 4000. Air from the RPT device 4000 is humidified in a humidifier 5000, and passes along an air circuit 4170 to the patient 1000. A bed partner 1100 is also shown.

FIG. 21B shows an RPT device 4000 in use on a patient 1000 with a nasal mask 3000.

FIG. 21C shows an RPT device 4000 in use on a patient 1000 with a full-face mask 3000.

FIG. 22 shows an example non-invasive patient interface 3000 in the form of a nasal mask.

FIG. 23A shows an RPT device 4000 in accordance with one form of the present technology.

FIG. 23B shows a schematic diagram of the pneumatic circuit of an RPT device 4000 in accordance with one form of the present technology. The directions of upstream and downstream are indicated.

FIG. 23C shows a schematic diagram of the electrical components of an RPT device 4000 in accordance with one aspect of the present technology.

FIG. 23D shows a schematic diagram of the algorithms 4300 implemented in an RPT device 4000 in accordance with an aspect of the present technology. In FIG. 23D, arrows with solid lines indicate an actual flow of information, for example via an electronic signal.

FIG. 23E is a flow chart illustrating a method 4500 carried out by the therapy engine module 4320 of FIG. 23D in accordance with one aspect of the present technology.

FIG. 24 shows a humidifier 5000.

### DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

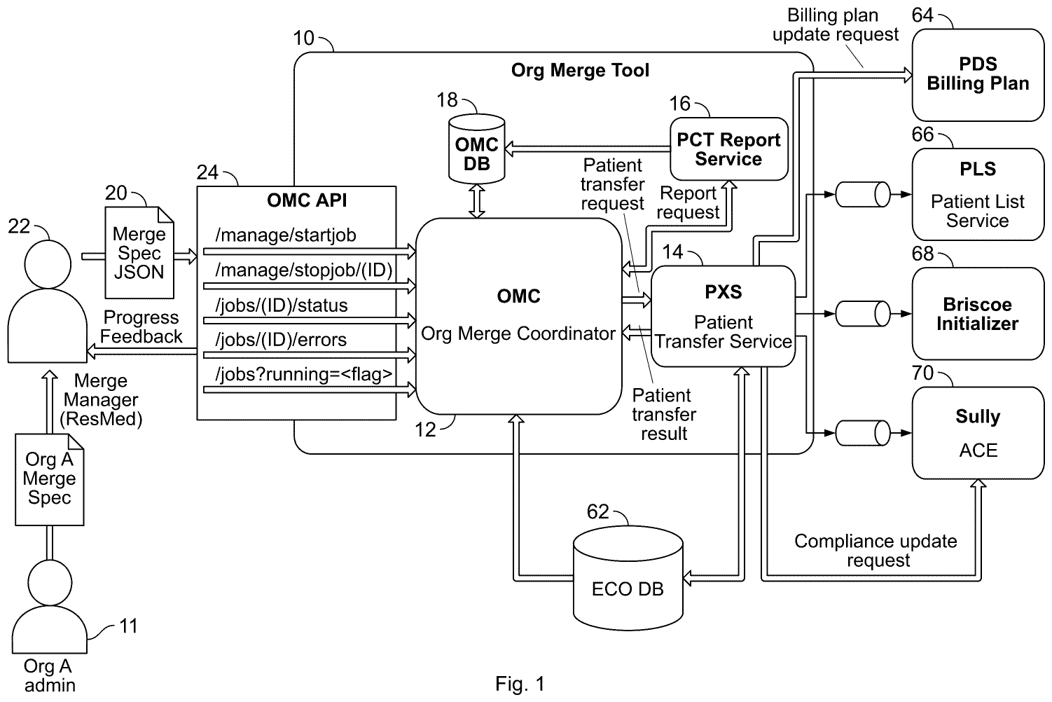

FIG. 1 illustrates a flow of example operations of a patient consolidation tool 10 for a patient data management system for transferring access to patient data from one organization to another organization, according to the present technology. The need for such a consolidation could be initiated by different reasons. For example, merger and acquisition between organization can require such data transfers. Similarly, a single patient wanting to transfer to another organization can require a data transfer. With the present technology, providers can become more productive by providing access to newly acquired patients. For example, when an organization acquires or merges with another organization, the patient data of the acquired organization need to be merged or consolidated with the patient data of the acquiring organization for the internal systems to operate properly. Typically, the patient data of the acquired organization and the patient data of the acquiring organization are consolidated by transferring access to patient data of the patients from the acquired organization to the acquiring organization, such as after receiving a transfer request from an administrator 11 of an involved organization. In the present disclosure, the organization may be referred to as a therapy provider organization for respiratory disorders, such as a Home Medical Equipment (HME) company, or other medical organization such as physician organizations, sleep labs, or an integrated delivery network (IDN) (i.e., a network of healthcare providers and facilities within a specific geographic region that offers patient care). Such patient data may even be utilized for making changes to patient therapy, such as where the data is utilized to communicate control commands to a patient therapy device for making changes to the operation of the therapy device, such as a change to a therapy setting for a therapy provided by such a device. The patient data may be organized and maintained in the data systems and/or data services (e.g., server(s), list(s), and/or database(s)) of a management entity, in which the patient consolidation tool 10 operates. As discussed in more detail herein, the tool may be considered a set of one or more processes or services running on one or more processors, such as the processor(s) of a server(s). The processes may be implemented by software as discussed in more detail herein to enable the aforementioned transfer(s) with the data systems such as by generating communications and/or control commands to the data systems to perform the access transfer as well as receiving input and providing output for monitoring such transfers. Moreover, the system may record and track data regarding such consolidations and transfers of patient records such that analytics may be generated regarding trends in patient movement in and/or out of particular organizations.

The present technology provides several benefits over the conventional methods (i.e., customizing and executing stand-alone database scripts) of transferring access to patient data. For example, the tool 10 according to the present technology provides an efficient and effective solution to transfer access to patient data from one organization to another organization, thereby eliminating the issues caused by the complexity of a conventional method of transferring patient data process. In addition, the tool 10 may be configured to be operated in conjunction with a plurality of data storage systems used by a management entity to maintain consistent patient data across the plurality of systems, while readily permitting that data to be newly accessed by the acquiring organization. As such, the present technology provides a solution to address a problem associated with the complexity of organization and management of data maintained by and within digital data systems. Furthermore, when such transferred patient data includes therapy device data (e.g., device settings and/or a unique device identification) that may be utilized for making changes to the transferred patient's therapy provided by the patient's therapy device, completion of a transfer with the transfer technology disclosed herein permits a new organization to take control of management of operation of a transferred patient's therapy device based on the transfer, so as to permit the new organization to operate the patient data management system to electronically communicate with the transferred patient's therapy device. Such electronic communications may include sending data to, and/or receiving data from, the therapy device. Moreover, such communications may include communicating control commands from the system, such as via a network, to the transferred patient's therapy device(s) for remotely making changes to the operation of the therapy device(s), such as a change to a therapy setting for a therapy provided by such a device.

For example, as shown in FIG. 1, the patient consolidation tool 10 according to the present technology may include any one of more of a plurality of services 12, 14, 16, a database 18, and a user interface(s) 20. The tool 10 described herein is configured to transfer access to patient data related to patients of the source organization (e.g., an acquired organization) to the target organization (e.g., an acquiring organization), as will be described in greater details below. The components 12, 14, 16, 18, 20 of the tool 10 can be implemented with any combination of hardware or software, including software executed by multiple computer systems or servers.

The tool 10 may be configured to generate the user interface(s) 20 for communicating (input/output) with a user (e.g., a management entity administrator) 22 regarding the communications of the tool 10 with the plurality of services 12, 14, 16. Thus, the user interface 20 may be configured to receive user input needed for the plurality of services 12, 14, 16 to perform the patient transfer process and also transmit system output for the user to view, such as regarding status of such transfers. For example, the user interface 20 may be configured to receive user input data required by the plurality of services 12, 14, 16 to generate and execute a transfer job (i.e., a collection of one or more patient transfers) and also output the transfer job details for the user 22 to access. The user interface 20 may be a graphic user interface configured to permit visualized data input and provide data presentation. For example, the user interface may include drop-down menus and/or pre-determined selection options for quick entry and access.

In some implementations, the user interface 20 can be implemented by application(s) running on any of a web-based platform (online/Internet web application of a server) and/or a mobile-based platform (mobile application), such that the user may access the user interface of the tool, and/or communicate with it, over a network, such as an internet or the Internet, using a computing device that includes a display, an input device and a communications interface. Non-limiting examples of computing devices include a personal computer (laptop or desktop), mobile phone (smartphones), tablets, personal digital assistants (PDA), or other similar devices. The computer devices will typically access the system directly through an Internet service provider (ISP) or indirectly through another network interface.

Referring again to FIG. 1, the plurality of services 12, 14, 16 facilitate creating a transfer job based on the user input and executing the transfer job by communicating with other systems that maintain the patient data, to transfer the access to the patient data associated with the transfer job from the source organization to the target organization. The plurality of services may include an organization merge coordinator (OMC) service 12, a patient transfer (PT) service 14, and a report service 16. The OMC service 12 is configured to communicate with the user interface 20, or through the user interface 20 such as when the OMC is configured to generate the user interface. In some implementations, the OMC may be implemented with an application programming interface (API) 24 that is configured to receive requests from the user interface 20 to facilitate communication with the OMC service 12 and provide responses as output to the user interface 20. For example, once the user 22 makes a certain input action (e.g., clicking a button or icon embedded on a webpage) on the user interface 20, the user interface 20 can generate a communication as a request for any of: data retrieval (i.e., retrieving transfer job details), data storage (i.e., storing transfer job details), and/or the like, and communicate the request to the API 24. The OMC service 12, via the API 24, may then interpret the request and act on the request. In some implementations, the request sent by the user interface 20 can be calls made via XML over hypertext transfer protocol (HTTP), calls made directly to the API 24, and/or calls made to a wrapper around the API 24.

As previously noted, the user interface 20 may be operated by a user to enter job transfer input data. The transfer job input data is information needed by the OMC service 12 to perform a patient transfer process. The transfer job input data may include transfer information related to the source (or acquired) organization and the target (acquiring) organization. Such input data provided by the user 22 may be entered and sent or communicated to the API 24, for example and without limitation, as, JSON, XML data or the like and may be communicated by a suitable communications protocol, such as HTTP. Optionally, JSON (a data interchange format) may be used by the user interface to format the input data provided by the user 22 as a specification for a merge of patient data between two organizations or a “merge specification,”

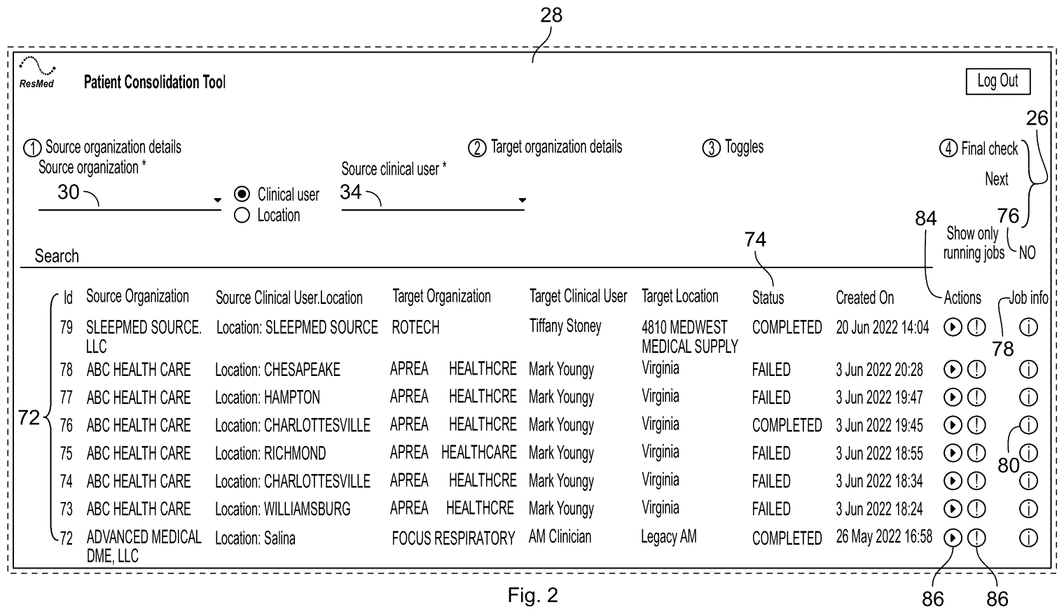

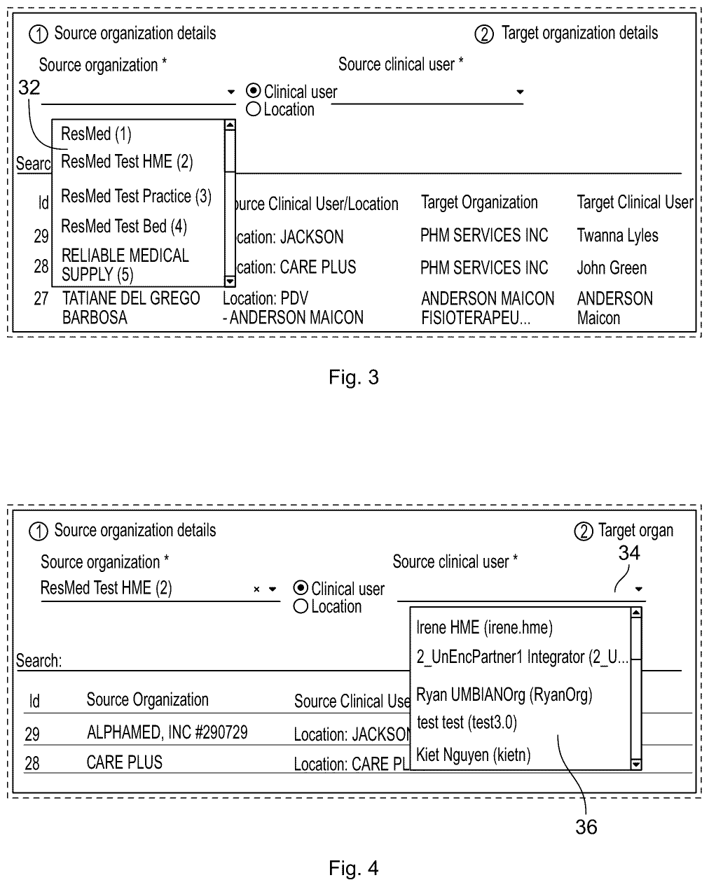

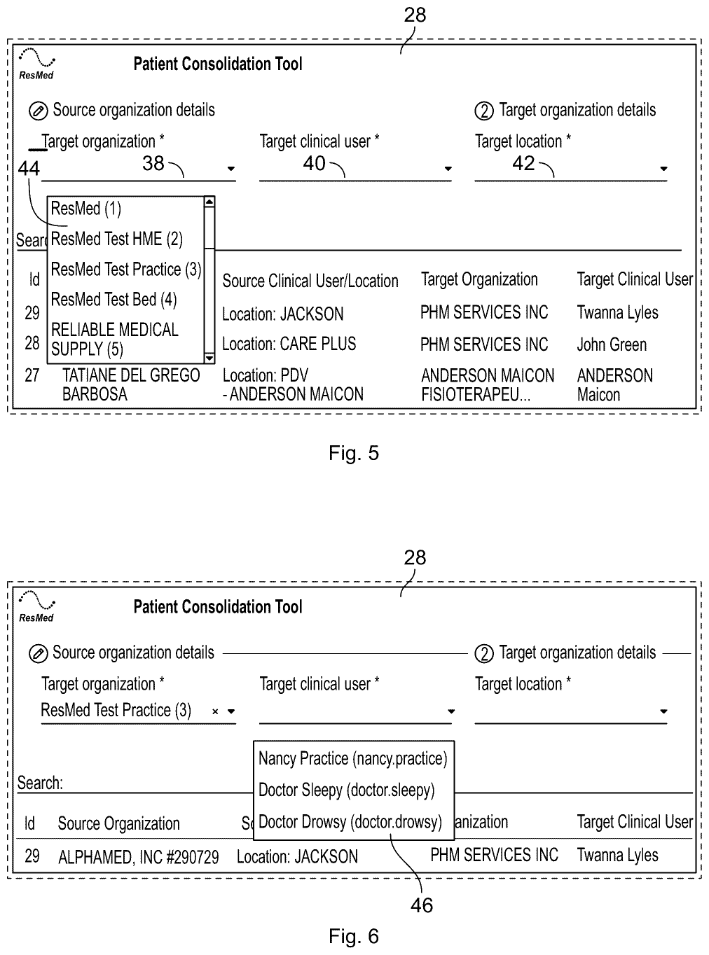

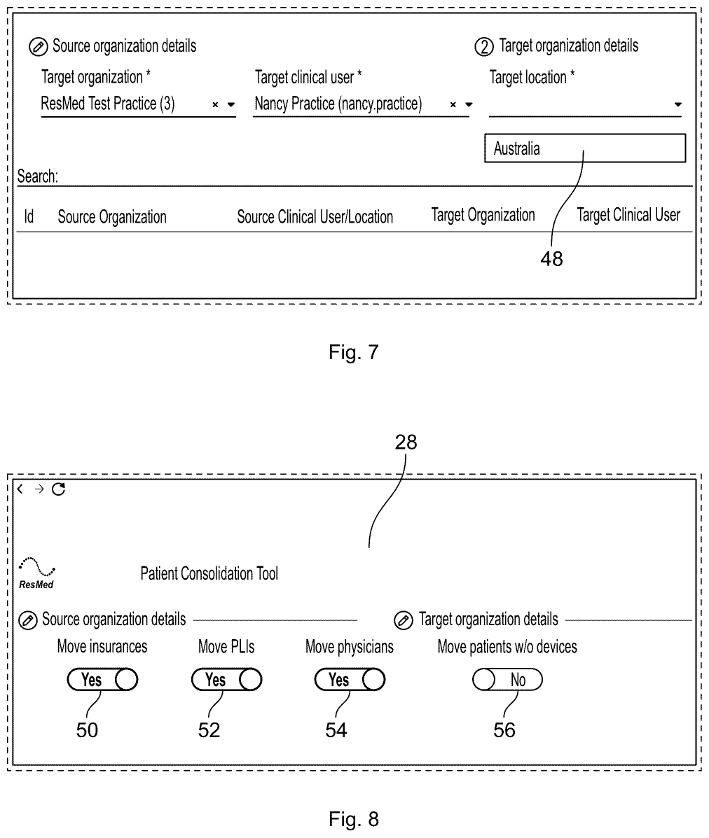

Thus, the user interface 20 may be configured for entering a merge specification for a transfer job, which may include the input of the transfer information related to the source and target organizations that will merge patients from the source organization into the target organization so that the target organization obtains access to the merged patients data. Specifically, as shown in the example of FIG. 2, a transfer job creation section 26 of a home page 28 allows the user 22 to input various information related to the source and target organizations to create a transfer job. To do so, the user 22 provides the transfer information related to the source organization and the target organization. For example, the source organization field 30 is provided, which may be populated by selecting the source organization from the source organization listbox 32, as shown in FIG. 3. Moreover, grouping criteria may be selected with the user interface for targeting a subset of patients of the source organization that can be included in the merge or transfer specification. For example, the user interface may be configured to make selection to include patients associated with a particular location(s) or particular clinician(s). Similarly, in some implementations, the user interface may be configured to make a selection to include patients associated with particular devices (e.g., device type, such as any one or more of ventilators, sleep diagnostic screeners, and/or CPAP devices, or particular device model, etc.) Other criteria may also be implemented. In the example of FIG. 4, the source organization clinical user field 34 and the source organization location field (not shown) may either be populated by selecting the source organization clinical user from the source organization clinical user listbox 36 and/or the source organization location from the source organization location listbox (not shown), respectively. Similarly, the target organization field 38, target organization clinical user field 40, the target organization location field 42 may be populated by selecting the target organization from the target organization listbox 44, the target organization clinical user from the target organization clinical user listbox 46, and the target organization location from the target organization location listbox 48, respectively, as shown in FIGS. 5-7. In this regard, the user interface may be configured to enter a merge specification that may specify patient transfer on a location basis (i.e., all patients at a particular location of a source organization) and/or on a clinician basis (i.e., all patients managed by a particular clinician of a source organization). (See, e.g., FIGS. 2-4). Moreover, the user interface may be configured to enter a merge specification that may specify a particular target location for the transfer patients and/or a particular target clinician for the transfer patients. (See, e.g., FIGS. 5-7).

When creating the merge specification, the user may additionally specify particular types of information for transfer. Thus, the user interface may be configured to input one or more types of data that may be included or excluded from the transfer. For example, the user 22 may opt to retain (or transfer) various information related to the patients during the patient transfer process. For example, as shown in FIG. 8, the transfer creation section 26 may provide one or more selectors (e.g., check box or toggles), such a four toggle switches for transfer/updating of insurance information, physician information, as well as including patients that have not, or not yet, been assigned a home medical equipment, among others. Examples of such selectors are shown in FIG. 8 as “Move insurances” 50; “Move PLIs” 52; “Move physicians” 54; and “Move patients w/o devices” 56. However, the tool 10 may be adapted to incorporate as many toggle switches to retain additional information related to the patient as needed or desired for achieving a particular transfer in light of the nature of the data and the nature of the storage of such data by the data system(s).

Thus, these example toggle switches 50, 52, 54, 56 may be activated by user 22 to retain (or transfer) the information related to the toggle switch during the execution of the transfer job. For example, the user 22 may turn the insurance toggle switch 50 on to move the insurance information (e.g., insurance company name, insurance member id, etc.) related to the patients of the source organization to ensure that the patients are monitored correctly for compliance once they are transferred to the target organization. Similarly, when the physicians toggle switch 54 is on, the physician information (e.g., physician name, office address, etc.) related to the patients of the source organization is moved during the execution of the transfer job to ensure that each patient's physician link is retained. The patient without devices toggle switch 56 allows the patients without a therapy device (e.g., CPAP device) to be included during the patient transfer. The PLI (patient level integrator) toggle switch 52 may be implemented to ensure that the patient level integrations from the source organization are retained in the target organization.

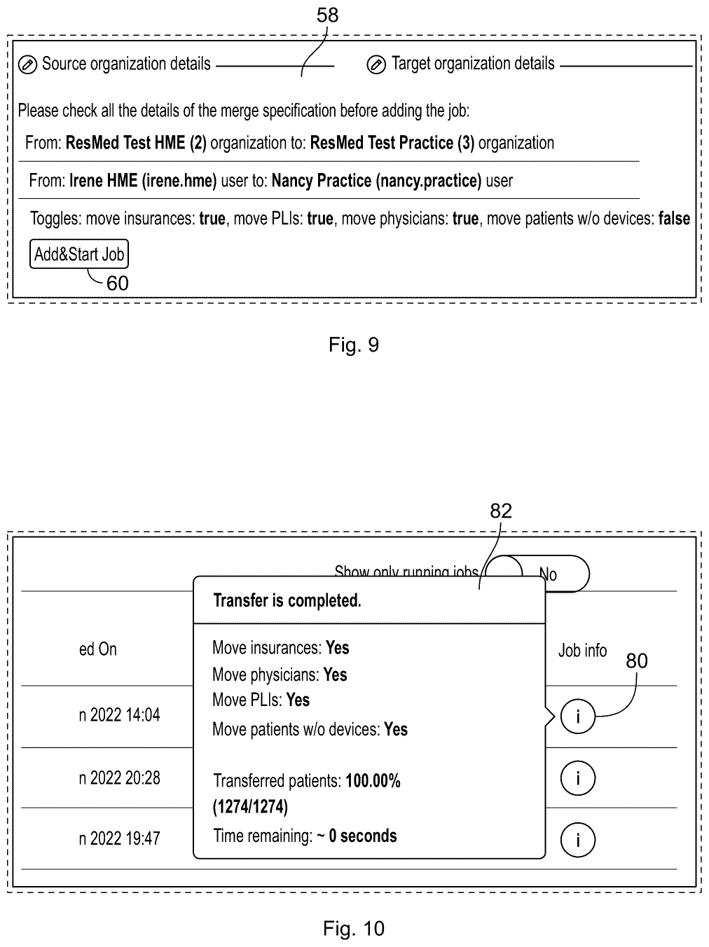

Once all the transfer information indicating the source organization and the target organization, including the information related to the toggle switches, are provided by the user 22, the user interface 20 displays a merge specification details page 58 for the user 22 to review the details of the transfer job information, such as in the manner of the example shown in FIG. 9. Upon clicking an input icon, such as the “Add& Start Job” button 60, the user input information (a merge specification) may be validated via a pre-transfer validation module, such as by checking one or more rules for such merges. The user input information and may then be communicated to the API 24 that will in turn transmit a request to the plurality of services 12, 14, 16 to generate and execute a transfer job based on the user input. In the event of a validation check, if the user input information fails the pre-transfer validation, the transfer job will not be generated, and thus the transfer job will not be executed. For example, if the source organization information provided by the user 22 is the same as the target organization information provided by the user, the pre-validation fails and the user 22 will be directed to an error page that displays the details of the errors. Once the user input information passes the pre-transfer validation, the user input data is formatted as a merge specification and the merge specification is transmitted to the API 24 to make a transfer command with the plurality of services 12, 14, 16.

Referring again to FIG. 1, the OMC service 12 is utilized to interpret and process the request from the API 24 to complete a transfer job based on the merge specification. The OMC service 12 may be operatively connected to and in communication with one or more databases, such as a OMC database 18 and central database (e.g., ECO database) 62. The central database 62 stores and maintain the patient data including the relationship (or links) between patients and organizations. Thus, the central database 62 includes a plurality of tables having multiple disparate data fields, and each of these data fields may include data pertaining to patients (e.g., demographic information, diagnosis information, therapy information, device information, compliance information, etc.), organizations (e.g., organization name, organization location, organization clinical user, etc.), etc.

Upon interpreting and processing the transfer command, the OMC service 12 may access the central database 62 to retrieve any necessary data to fulfill the request. For example, the OMC service 12 may determine which of the patients of the database are to be transferred (i.e., the patients that are associated with the source organization as specified by the conditions of the merge specification) by accessing (e.g., by generating one or more communications therewith) the central database 62 and retrieving the necessary data related to the patients of the source organization. Thereafter, the OMC service 12 organizes the retrieved data and creates a patient transfer dataset for the access transfer for each of the patients encompassed by the merge specification. Subsequently, the OMC service 12 generates a transfer job based on the patient transfer dataset and starts to execute the transfer job by issuing one or more requests to the PXS service 14 that includes instructions to execute transferring access to patient data of each patient included in the patient transfer dataset. For example, the OMC service 12 iterates through the patient transfer dataset and, for each patient in the dataset, issues the instructions to the PXS service 14 to affect transferring access to patient data of a patient until the last patient in the patient transfer dataset has been completed.

Thus, the PXS service 14 is operatively connected to and in communication with the OMC service 12, the central database 62 and one or more additional data systems or data services (e.g., listing services) that manage/organize storage of the patient data. Thus, the PXS service 14 is able to receive the request from the OMC service 12 and execute the instructions to perform transferring access of patient data of a patient at a time. For example, upon interpreting and processing the request from the OMC service 12, the PXS service 14 accesses the central database 62 to retrieve for data related to the patient, and assigns (or updates) the retrieved data with the target organization information (e.g., target clinical user, target location, etc.) specified in the merge specification such that the patient is now associated with the target organization.

Thus, the PXS service 14 is also operatively connected to and in communication with additional systems such as a plurality of systems 64, 66, 68, 70 that may be various data services. As such, each of the plurality of systems 64, 66, 68, 70 may provide further data that may depend on the patient-organization relationship. Thus, in order for the systems 64, 66, 68, 70 to operate properly with regard to the patient data of the central database 62, each system will be updated in light of patient-organization transfer as stored in the central database 62. Thus, after successfully transferring access to patient data associated with the patient in the central database 62, the PXS service 14 transmits a request to each of the plurality of internal systems 64, 66, 68, 70 to update the system with regard to the patient data of a transfer so that the system reflects the updated patient-organization relationship in the central database 62. Such updating may for example, involve compliance systems, billing systems, data metric systems, listing service(s), etc. For example, the following example systems may be updated based on the instructions received from the PXS service 14: PDS billing system 62; patient list service (PLS) system 66; Briscoe system 68; and Sully (ACE) system 70.

For example, one system may be a PDS billing system 64 that stores and manages billing plan information associated with patients. Thus, when the PXS service 14 sends an update request, the billing plan stored in the PDS billing system 64 are updated to reflect the updated billing plan associated with the transferred patients stored in the central database 62. If the billing plan for any of the transferred patient is not supported by the target organization, a new compatible billing plan may be created for such patient and stored in the PDS billing system 64 such that bills may be continued to generate for the patient. The physician associated with the transferred patient is also updated to maintain the patient-physician relationship stored in the central database 62.

Another example system may be a listing service, such as PLS system 66, that may store a list that is dependent on relationships between patients and organizations. Thus, updated patient-organization relationships related to transferred patients may be applied in the PLS system 66 by the communications with the PXS service 14. The updated listing of the PLS system 66 may then assist with allow the transferred patients to be accurately accessed by the target (new) organization, rather than the previous (old) organization.

Another example system may be a Briscoe system 68 that manages particular patients that use a common subset type of the HME devices of the central database, such as ventilators, and stores relationships between such patients (e.g., ventilator patients) and organizations. The updated patient-organization relationship related to the transferred patient may be applied in the Briscoe system 68 by the communications of the PXS service 14, such if the patient has a particular HME device of the common subset of device type (e.g., the patient is a ventilator user). For example, the Brisco system may be considered a device type subset data system that manages access, such as with a listing service, to particular display pages for patients to provide access to different data and different metrics that are uniquely associated with a subset of devices managed by the system. Such a system may, for example, manage access to pages and data for ventilation patients that have different data and different metrics compared to other device users (e.g., for user of ventilators rather than for users of CPAP devices or vice versa.)

Like the PLS system 66, the updated patient-organization relationships in the Briscoe system 68 may allow certain data of the transferred patients associated with the particular type of HME device to be accurately accessed by the target (new) organization, rather than the previous (old) organization.

Another example system may be a compliance system, such as the Sully system 70, that stores and maintains the data related to patient compliance rule(s). Insurance companies, or other reimbursing entities, often require evidence that the patient prescribed with respiratory therapy has been “compliant”, that is, has used their therapy device according to a predetermined “compliance rule” before reimbursing the patient for the therapy device. Compliance rules generally require some minimum amount of usage per session for some fraction of a number of consecutive sessions known as the compliance period. The compliance rules stored in the system 70 may be updated to reflect the updated compliance rule(s) associated with the transferred patients stored in the central database 62. While the central database 62 may store basic level of information related to the compliance rule(s) associated with each patient, compliance rule details may be stored in the additional system 70.

Upon completing each transfer job of a patient, the PXS service 14 communicates back to the OMC service 12 notifying that the particular patient transfer job has been completed. The OMC service 12 may then send the information related to the transfer job and the transferred patients (e.g., transfer job information, updated patient information, etc.) to the OMC database 18 for storage. The OMC database 18 is configured to store data structures corresponding to transfer jobs and transferred patients. For example, the OMC database 18 may store data such as transfer job id, source organization, target organization, job status, job creation date, patient id, etc. The OMC database 18 may be configured with any type of database such as a relational database, a distributed database, an object database, an object-relational database, NoSQL database, etc. The OMC database 18 may then be used (accessed) by the OMC service 12 to provide information to the user interface, such as in response to a request for status of a merge specification generated with the user interface.

For example, if an error(s) occurs during the patient transfer process performed by the PXS service 14, the service stops transferring access to patient data for the patient and communicates to the OMC service 12 that the error has occurred by returning the error details. The OMC service 12 may then display such information in the user interface 20. As previously noted, the OMC service 12 may continue to send instructions to the PXS service 14 to execute transferring access to patient data for each next patient in the patient transfer dataset from a merge specification until all patient have been processes from the dataset.

FIGS. 2, 10, and 11 are example webpages that may be used to access and manage the transfer jobs submitted to the OMC service 12. These webpages may be presented to the user, such as a merge manager, via the user interface 20 described above. FIG. 2 illustrates an example that may be a home page 28 of the system after the user 22 logs into the system with the established authentication credentials (e.g., username and password). Such a home page 28 may allow the user 22 to view a list of transfer jobs that are stored in the OMC database 18. As shown in FIG. 2, the transfer job page may include multiple sections such as: a transfer job information section 72 for displaying a list of transfer jobs and a transfer job creation section 26 for allowing the user to input information related to the source and target organizations to generate a transfer job. The transfer job creation section 26 operates as previously described.

As illustrated in the example of FIG. 2, the transfer job information section 72 may display a list of transfer jobs, such as with a plurality of columns that display information such as source organization, source clinical user, source clinical location, target organization, target clinical location, target clinical user, and/or status, etc. The status column 74 indicates the status of the transfer job. For example, the “running” status indicates that the job is currently being executed, “completed” status indicates that the job is completed without any errors, and “failed” status indicates that the job is completed with errors. For user's convenience, the list of transfer jobs may be filtered so as to only display “running” jobs or “completed”/“failed” jobs, such as by filtering the transfer jobs using an input selector such as a job toggle switch 76.

The transfer job information section 74 of the home page 28 also provides input elements (e.g., buttons, icons, etc.) for the user 22 to activate to access and manage the transfer jobs that are displayed thereon. Each input element can request information related to the function of the input element by generating a call to the API 24 upon activation of the input element, which may optionally in turn communicate with any of the plurality of services 12, 14, 16 to formulate and return the desired result. For example, as can be seen in FIG. 2, the “job info” column 78 displays a clickable “i” icon 80. When the icon 80 is clicked by the user, the OMC service 12 receives a request from the API 24 and queries the OMC database 18 to obtain the details pertaining to the clicked transfer job saved in the OMC database 18. Then, the user interface 20 generates a pop-up panel 82 containing the retrieved transfer job details (e.g., number of patients transferred, etc.) thereon, as shown in the example display of FIG. 10.

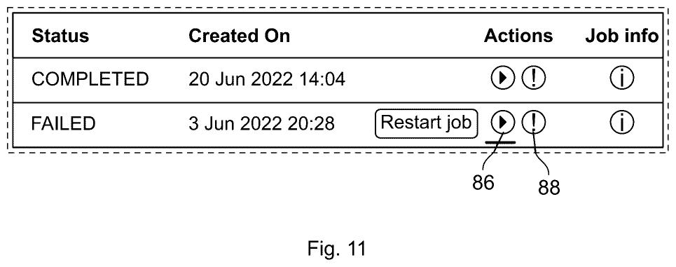

In addition, the “action” column 84 provides various action icons that the user 22 may activate (e.g., click) to perform a specific action to manage the transfer job. For example, if the user 22 wishes to stop a transfer job that is currently running, the user 22 may click on the “square” icon (not shown) and a call to the OMC API will be generated so that the running transfer job will be stopped. For the “failed” job, the “arrow” icon 86 and “exclamation mark” icon 88 are provided in the action column 84. As shown in FIG. 11, the arrow icon 86 allows the user 22 to restart the failed transfer job, whereas the exclamation mark icon 88 directs the user 22 to the error page that displays the details of each error occurred during the execution of the transfer job. Thus, the OMC service 12 may receive a request from the API 24 once a specific icon is activated (e.g., clicked) in light of the purpose of the specific icon. Thus, for the icon 88, upon processing the request by the API 24, the OMC service 12 may query the OMC database 18 to obtain the error details pertaining to the clicked transfer job saved in the OMC database 18 and respond with detail information for display in/with the user interface 20. As for the completed job shown in the example of FIG. 11, the arrow and exclamation mark icons 86, 88 may be disabled by the user interface 20 since the transfer job is completed without any errors.

The user interface 20 may be configure to enable a user 22 to download and view the details of the completed transfer job by activating (e.g., clicking) the “downward arrow” icon (not shown) provided by the user interface 20. For example, when the user 22 clicks on the icon, the OMC service 12 receives a request from the API 24 and sends instructions to a report service 16 to query the OMC database 18 to obtain the data related to the transfer job. Thereafter, the report service 16 may generate a readable file, such as with formatted data, (e.g., PDF or Excel) that includes the retrieved data and transmits the file to the user's computing device for the user 22 to access.

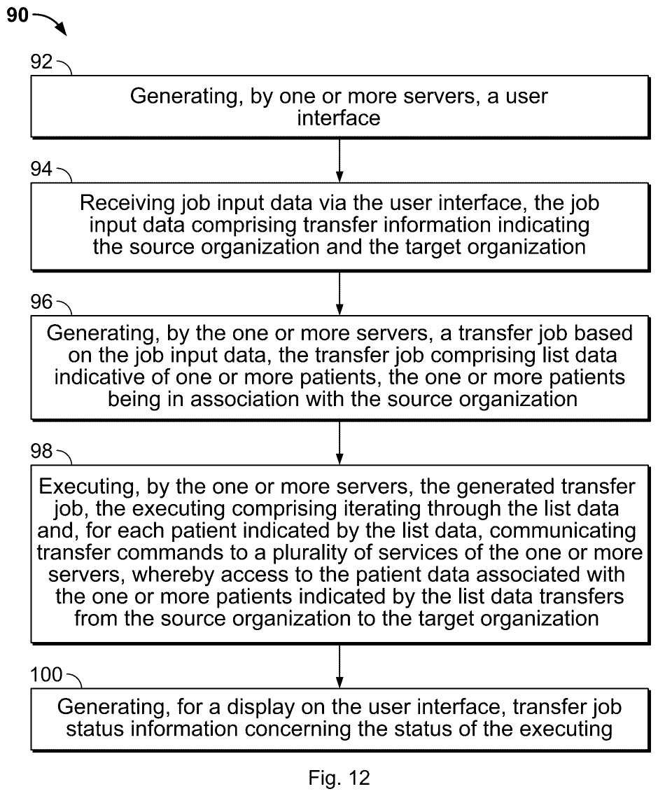

Accordingly, the present technology may implement systems that permit streamlined patient data transfers. For example, with reference to FIG. 12, a method 90 of such a system may permit transferring access to patient data from a source organization to a target organization. In this example process of FIG. 12, at step 92, the method may include generating, by the one or more servers, a user interface. At step 94, the method may include receiving job input data via the user interface. The job input data may include transfer information indicating the first organization and the second organization. At step 96, the one or more servers may generate a transfer job based on the job input data. The transfer job may include list data indicative of the one or more patients. The one or more patients may be in association with the first organization. At step 98, the one or more servers may execute the generated transfer job. The executing may include iterating through the list data and, for each patient indicated by the list data, communicating transfer commands to a plurality of services of the one or more servers. As such access to the patient data associated with the one or more patients indicated by the list data transfers from the first organization to the second organization. At step 100, the method may include generating, for a display on the user interface, transfer job status information concerning the status of the executing.

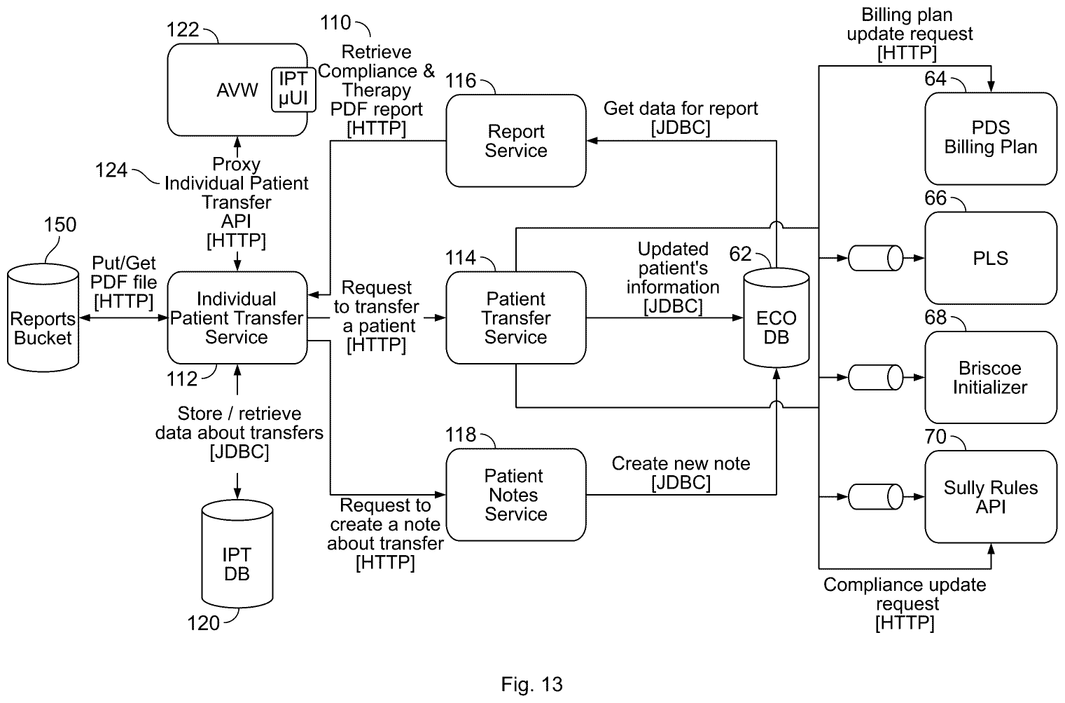

FIG. 13 illustrates an individual patient transfer system 110 for transferring access to patient data associated with a particular patient from one organization (e.g., therapy provider organization) to another organization (e.g., therapy provider organization), according to another example of the present technology. Similar to the patient consolidation tool 10 described above, the individual patient transfer system 110 includes a plurality of services 112, 114, 116, 118, a database 120, and a user interface 122. The individual patient transfer system 110 described herein is configured to transfer access of patient data for a particular patient, as will be described in greater details below. The components 112, 114, 116, 118, 120, 122 of the system can be implemented with any combination of hardware or software, including software executed by multiple computer systems or servers.

The user interface 122 is provided by the system, such as by the AVW service of a device or server(s) of the system, for communicating between a user (e.g., data administrator) and the plurality of services 112, 114, 116, 118. The user interface 122 serves to collect user input data needed for the plurality of services 112, 114, 116, 118 to perform the patient transfer of a patient and also serves to transmit system output for the user to view. For example, the user interface 122 may be configured to capture user input data required for the plurality of services 112, 114, 116, 118 for transferring access to patient data of the patient and also output the patient transfer details stored in the database 120. The user interface 122 may be a graphic user interface, which allows easy data input and data presentation. The user interface 122 may include data selectors such as drop-down listbox and other selectors for quick entry and access.

The user interface 122 can be implemented as a stand-alone application on both a web-based platform (online/Internet web application) and mobile-based platform (mobile application) such that the user may access it over the Internet using a computing device which includes a display and an input device implemented therein. Non-limiting examples of computing devices include a personal computer (laptop or desktop), mobile phone (smartphones), tablets, personal digital assistants (PDA), or other similar devices. The computer devices will typically access the system directly through an Internet service provider (ISP) or indirectly through another network interface.

Referring again to FIG. 13, the plurality of services 112, 114, 116, 118 facilitate transferring access to patient data of the patient (one at a time) from a current organization to a target (or destination) organization. The plurality of services include an individual patient transfer (IPT) service 112, a patient transfer (PT) service 114, a report service 116, and a patient notes (PN) service 118. The IPT service 112 is operatively connected to and in communication with each of the PT service 114, report service 116, and PN service 118. The IPT service 112 is also operatively connected to and in communication with the user interface 122. Specifically, an API 124 can be utilized to interpret and process requests from the user interface 122 to facilitate communication with the IPT service 112. For example, once the user takes a certain action (i.e., clicking a button or icon embedded on a webpage), the user interface 122 can specify a request for data retrieval (i.e., retrieving transfer patient details), data storage (i.e., storing transfer patient information in IPT database), and/or the like to the API 124. Thereafter, the API 124 can interpret the request, and the API 124 transmits a transfer command to the IPT service 112 to perform a particular function. The input data provided by the user can be captured and sent to the API 124 as a request, for example and without limitation, JSON, XML and the like, and may be communicated by a suitable communications protocol, such as HTTP. In the present disclosure, JSON (a data interchange format) is used to format the input data provided by the user.

There are several steps that may be involved in fulfilling transferring access to patient data of a patient from a current organization to a target organization. One step may be to query the central database 62 to determine whether a patient record exists based on the device identification indicia of the device associated with the patient. Another step may be to transfer access of the patient data of the patient from the current organization to the target organization, such as if the patient record exists in the central database 62 as a result of the query.

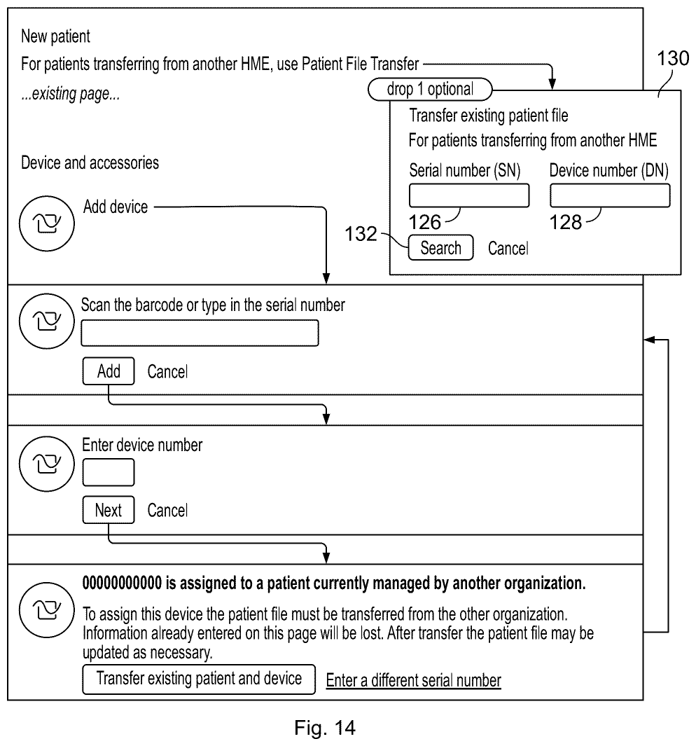

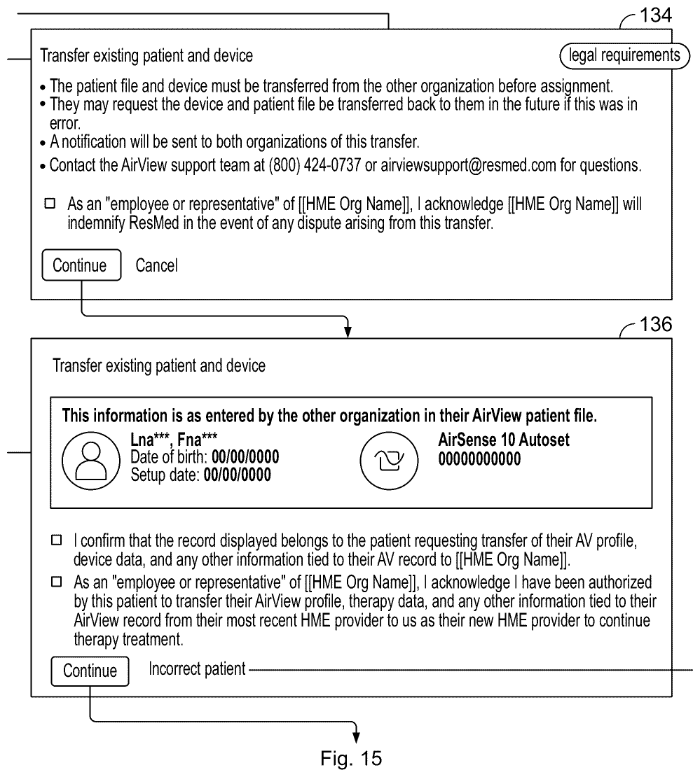

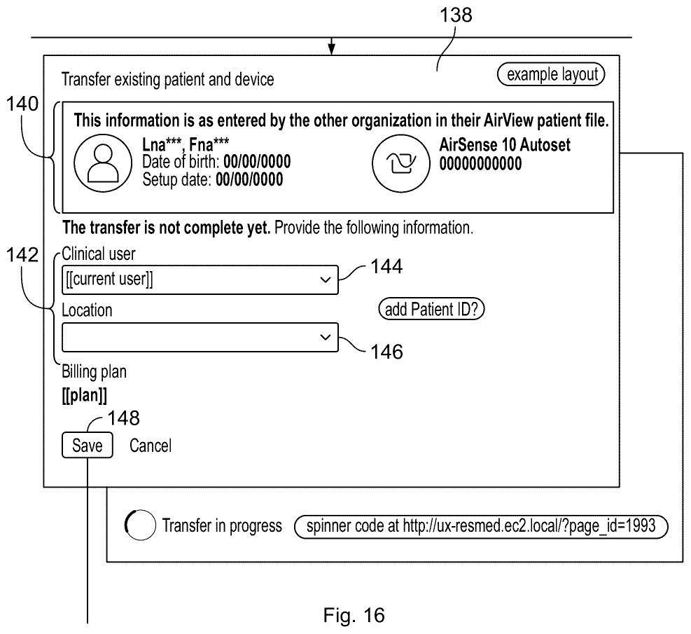

To affect the query step, the user may provide a serial number and/or a device number of the therapy device associated with the patient in the serial number field 126 and device number field 128 embedded in the webpage 130, respectively, as shown in FIG. 14. In some implementations, the query may require both (serial number and a device number) as a basis for validation or reducing errors. Additionally, or alternatively, entry of patient name may be required to advance the query. Thereafter, the user may click the “Search” button 132. When the Search button is clicked, the AVW service (which may provide the user interface 122) determines if the patient's device is currently being used by a patient in a different organization by querying the database (62). The IPT service 112 may then be used when the save button 148 shown in FIG. 16 is activated (e.g., clicked) to activate a transfer, after confirmation by the search. In this regard, the API 124 may transmit a transfer command with the input data (e.g., device identification indicia provided by the user) to the IPT service 112 after the AVW confirms the presence of the patient device with the AVW service. For the transfer, the user interface 112 may optionally present the user with the screens or pages for the user to consent. For example, the pop-up pages 134, 136, as shown in FIG. 15, may be presented such that the user may consent to release or indemnify a system provider in case of conflict and confirm the patient to be transferred. Upon consent by the user, the user interface 122 may redirect the user to an individual patient transfer page 138, which displays the patient information for the user to review, as shown in FIG. 16.

Thus, using the individual patient transfer page 138, the second step of the patient transfer process may be performed. The individual patient transfer page 138 includes two main sections: a patient information section 140 and a user input section 142. As shown in FIG. 16, the patient information section 140 displays the patient information, e.g., patient name, patient date of birth, etc., retrieved from the central database 62 during the first step of the patient transfer process.

The user input section 142 may be configured to capture the input data provided by the user. The input data may include the target organization clinical user and the target organization location, which may be provided by selecting from the target clinical user listbox 144 and target organization location listbox 146, respectively.

As previously noted, after the user input indicating the target organization are provided by the user, the user may click the “Save” button 148 to transmit the user input to the API for sending a transfer command to the IPT service to execute the patient transfer process.

The IPT service 112 may then perform similar functions as the OMC service 12 in the patient consolidation tool 10 as previously described. For example, the IPT service 112 communicates with the PT service 114 by processing and sending a transfer command received from the API 124 to the PT service 114. The PT service 114 then executes transferring access of patient data of the patient from the current organization to the target organization. Thus, the functions of the PT service 114 may be identical to those of the PXS service 14 of the patient consolidation tool 10 as previously described. For example, the PT service 114 may access and retrieve the central database 62 for the record related to the patient and update the retrieved record to reflect that the patient is now linked to the target organization. The PT service 114 may then transmit a request (or instruction) to each of the plurality of systems 64, 66, 68, 70 to update the record related to the patient to reflect the new patient-organization relationship for the patient. The plurality of internal system may include PDS billing system 64, patient list service (PLS) system 66, Briscoe system 68, and Sully system 70, which may be identical to the systems previously described in regard to the operations of the patient consolidation tool 10. After updating the central database 62 and the plurality of systems 64, 66, 68, 70, the PT service 114 may communicate to the IPT service 112 that the patient transfer process has been completed by returning the transfer details (e.g., patient information, target organization information, etc.) to the IPT service 112. Upon receiving the indication from the PT service 114, the IPT service 112 accesses the IPT database 120 to save and store the transfer details for the user to retrieve and review via the user interface 122. The IPT database 120 may be configured with any type of database such as a relational database, a distributed database, an object database, an object-relational database, NoSQL database, etc.

After completing the patient transfer process by the PT service 114, the report service 116 may access the central database 62 and retrieve information related to compliance rules and therapy associated with the transferred patient to generate a “compliance & therapy” report. The compliance and therapy report may be generated as a readable file, such as a PDF file, and be saved in a database such as a reports bucket 150 (shown in FIG. 13) by the IPT service 112 such that the users of the previous organization may be able to access and view via the user interface 122.

In addition, the PN service 118 may interpret and process the request received from the IPT service 112 and performs the necessary functions to complete the patient transfer process. For example, the PN service 118 may access the central database 62 and stores the user input information related to the legal consent (e.g., indemnification and patient transfer confirmation) gathered from the user interface 122. Then, the PN service 118 may generate a note using the consent information gathered from the user interface 122 and stores the note in the central database 68 for displaying on the user interface 122 when requested.

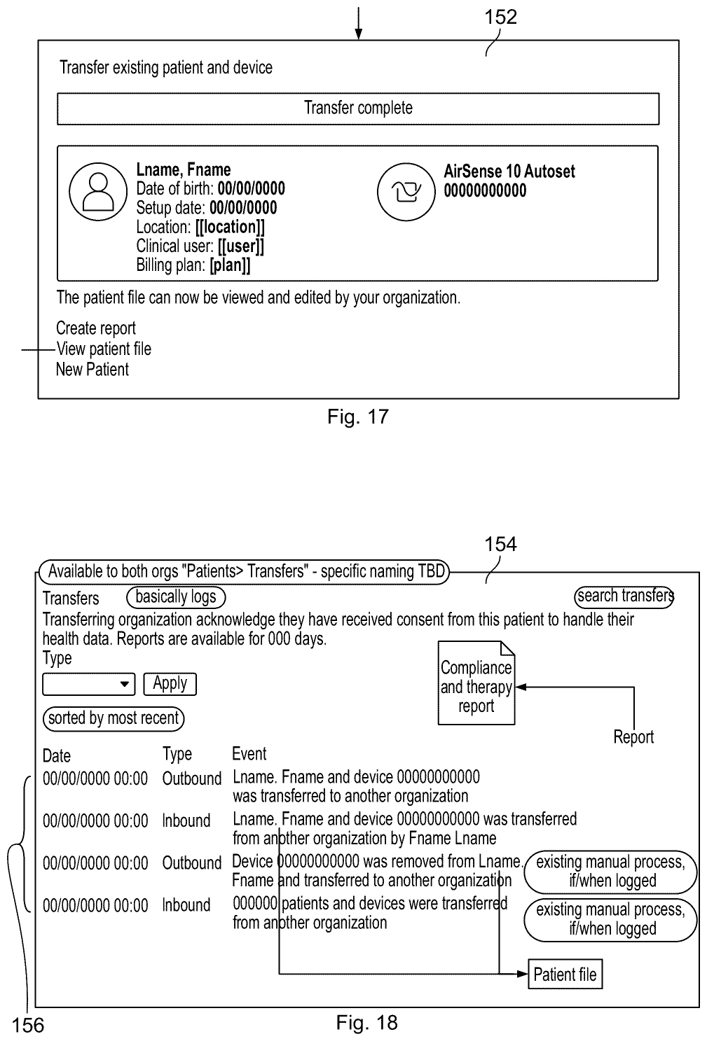

Once the patient transfer process completes, the user interface 122 may then be redirect the user to the confirmation page 152, as shown in FIG. 17, that includes the information related to the transferred patient. If an error(s) occurs during the patient transfer process performed by the PT service 114, report service 116, and/or PN service 118, the services stop the patient transfer process and communicate to the IPT service 112 that the error has occurred by returning the error details, which the user interface 122 may display or otherwise present to the user.

As illustrated in FIG. 18, the system may provide a user interface and associated functionality to permit display of a transfer page 154. Such a user interface display may be generated by the system to allow the user to view the transferred patients that are saved and stored in the IPT database 120. The transfer page 154 may display a list of transferred patients 156, such as with a plurality of sections or columns displaying information such as transfer type, transfer event, etc. For example, the transfer type column 158 indicates whether the patient is transferred in or transferred out. For example, the transfer type of “outbound” indicates that the patient is transferred out of the organization, whereas the transfer type of “inbound” indicates that the patient is transferred into the organization from another organization. Thus, if a user of the target organization accesses the transfer page 154, the patient that is indicated as “outbound” for the source organization will instead be indicated as the “inbound” patient for the target organization. Optionally, the transfer page may further include a user interface control, such as a button, so the user can trigger the system to export, such as to a file, a copy of the list of transferred patients.

In the transfer page 154, the user interface 122 allows the user to access only the profile of the “inbound” patients and thus access to the profiles of the “outbound” patients are denied. However, the transfer page 154 provides a feature such that the user may still view the information related to compliance and therapy associated with the “outbound” patient. For example, a report link 158 may be clicked to call the API 124 to transmit a request to the IPT service 112. Upon interpreting and processing the request, the IPT service 112 may access the reports bucket 150, retrieve a report related to the “outbound” patient, and display the report on the user interface 122 for the user to review.

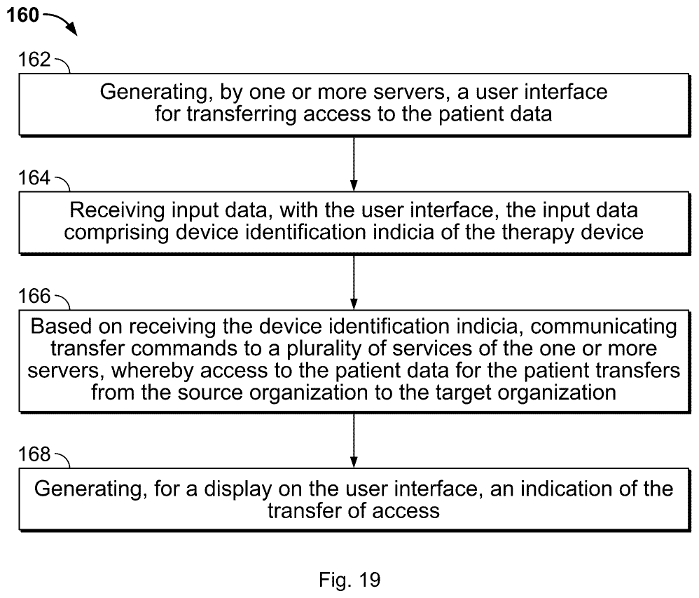

Accordingly, the present technology may implement systems that permit streamlined patient data transfers. For example, with reference to FIG. 19, a method 160 of such a system may permit transferring access to patient data from a source organization to a target organization In the example process of FIG. 19, at step 162, the method may include generating, by the one or more servers, a user interface for transferring access to the patient data. At step 164, the method includes receiving input data, with the user interface, the input data comprising device identification indicia of the therapy device. At step 168, the method includes, based on receiving the device identification indicia, communicating transfer commands to a plurality of services of the one or more servers, whereby access to the patient data for the patient transfers from the first organization to the second organization. At step 168, the method includes generating, for a display on the user interface, an indication of the transfer of access.

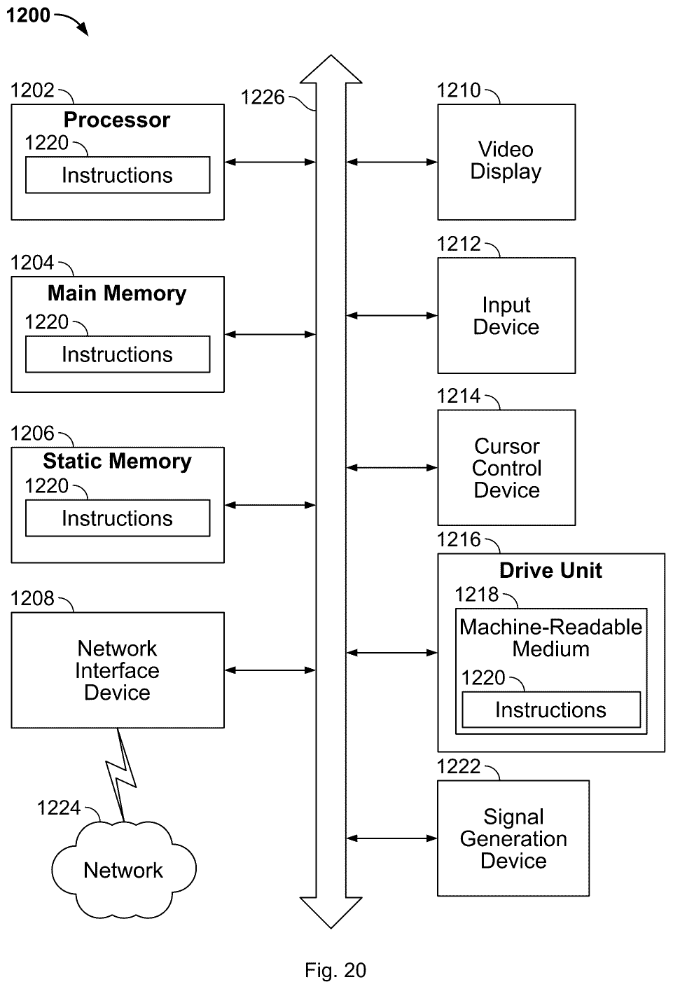

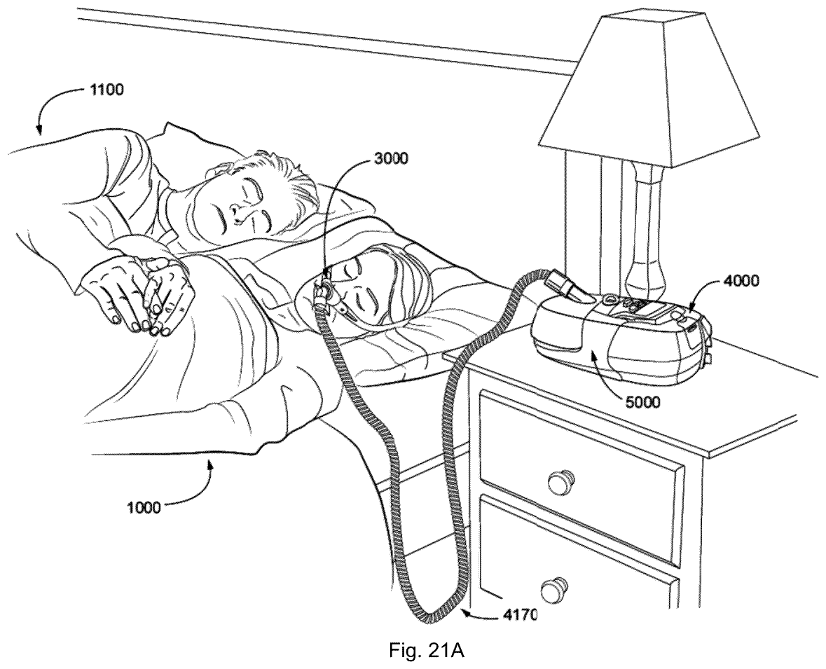

FIG. 20 is a block diagram of an illustrative example of a general computing system 1200. The systems of the present technology disclosed herein may be implemented using the computing system. In addition, the computing system 1200 can include a set of instructions that can be executed to cause the computing system 1200 to perform the methods disclosed herein. The computing system 1200 may operate as a standalone device or may be connected, e.g., using a network 1224 or other connection, to other computing systems or peripheral devices.

As shown in FIG. 20, the computing system 1200 may include a processor 1202, e.g., a central processing unit (CPU), a graphics-processing unit (GPU), or both. Moreover, the computing system 1200 may include a main memory 1204 and a static memory 1206 that can communicate with each other via a bus 1226. As shown, the computing system 1200 may further include a video display unit 1210, such as a liquid crystal display (LCD), an organic light emitting diode (OLED), a flat panel display, a solid state display, or a cathode ray tube (CRT). Additionally, the computing system 1200 may include an input device 1212, such as a keyboard, and a cursor control device 1214, such as a mouse. The computing system 1200 can also include a disk drive unit 1216, a signal generation device 1222, such as a speaker or remote control, and a network interface device 1208.

In a particular example or aspect, as depicted in FIG. 12, the disk drive unit 1216 may include a machine readable or computer-readable medium 1218 in which one or more sets of instructions 1220, e.g., software, can be embedded, encoded or stored. Further, the instructions 1220 may embody one or more of the methods or logic as described herein. In a particular example or aspect, the instructions 1220 may reside completely, or at least partially, within the main memory 1204, the static memory 1206, and/or within the processor 1202 during execution by the computing system 1200. The main memory 1204 and the processor 1202 also may include computer-readable media.

In accordance with various examples or aspects, the methods described herein may be implemented by software programs tangibly embodied in a processor-readable medium and may be executed by a processor. Further, in an exemplary, non-limited example or aspect, implementations can include distributed processing, component/object distributed processing, and parallel processing. Alternatively, virtual computing system processing can be constructed to implement one or more of the methods or functionality as described herein.

### Optional Example Treatment Devices

As previously noted, patient data managed by the aforementioned access transfer systems may include data concerning use and/or operation of a home medical equipment or respiratory therapy device. Examples of such devices may be considered in relation to the examples discussed in more detail in the following sections.

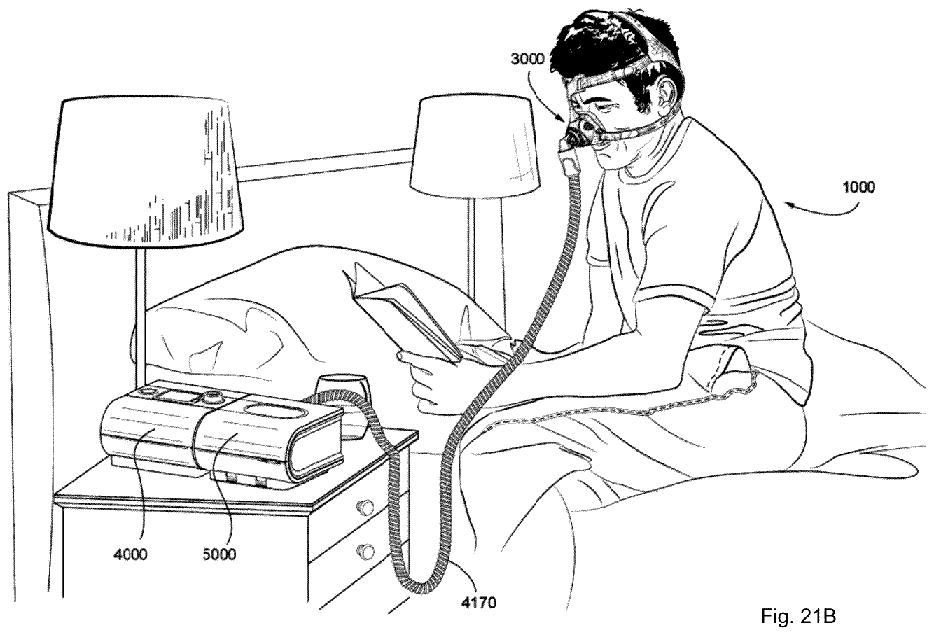

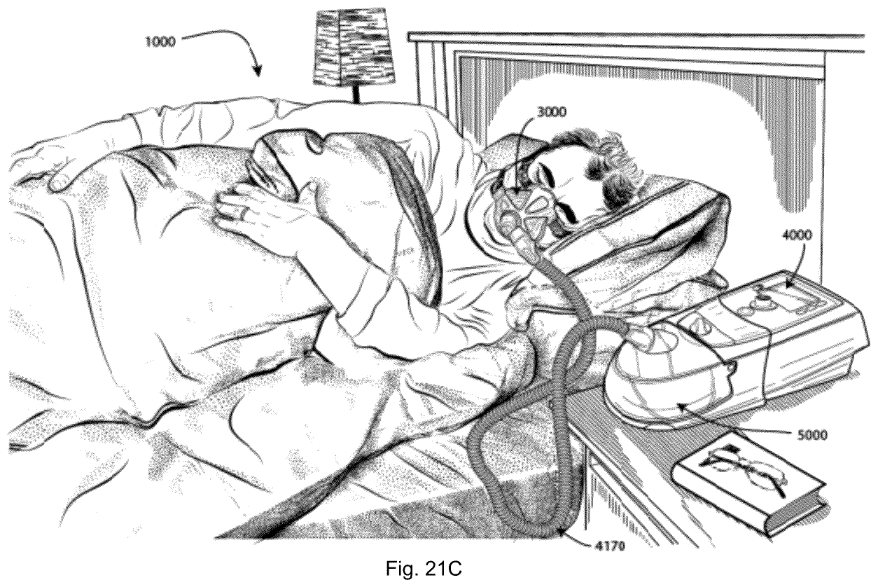

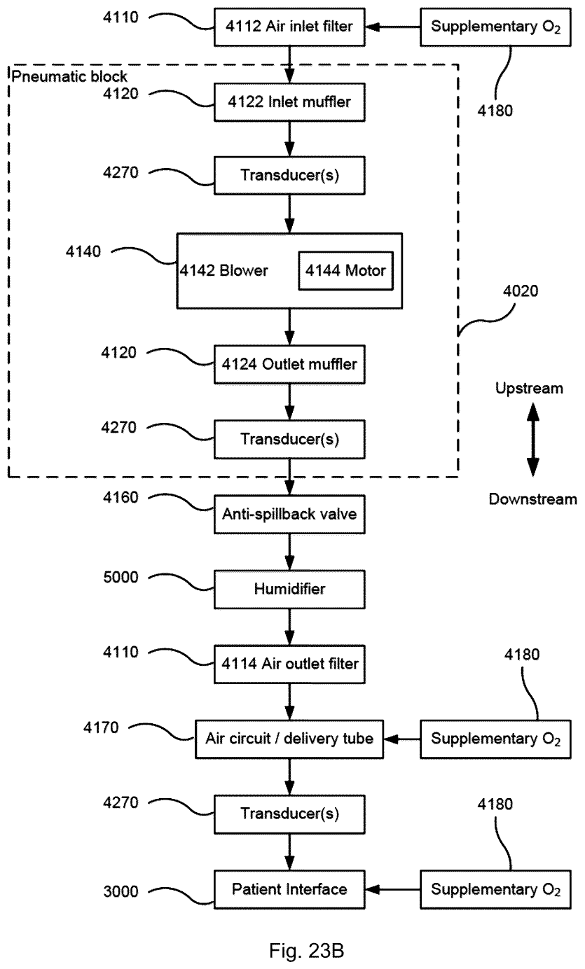

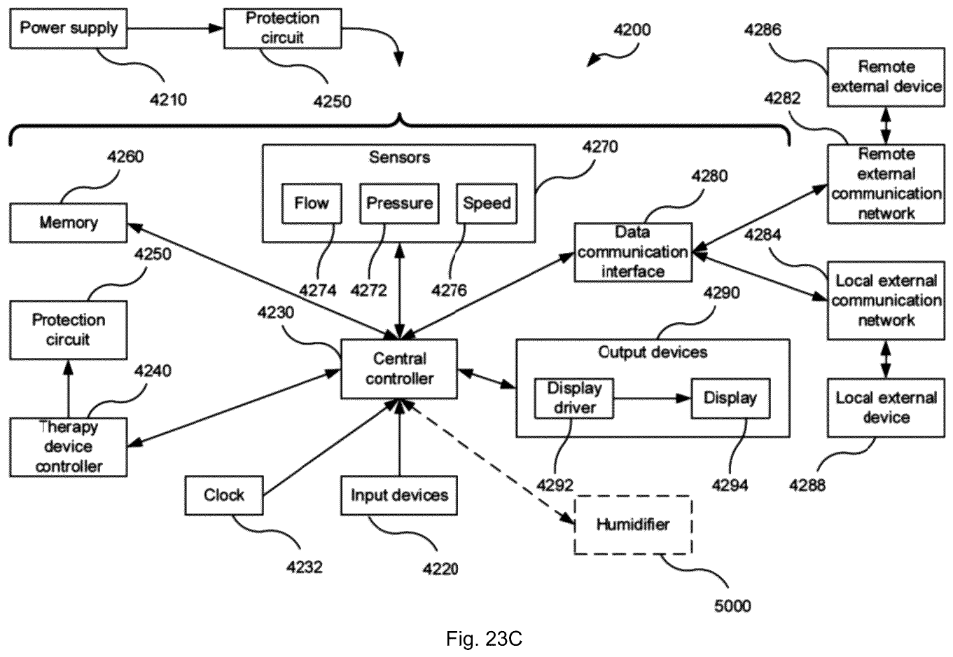

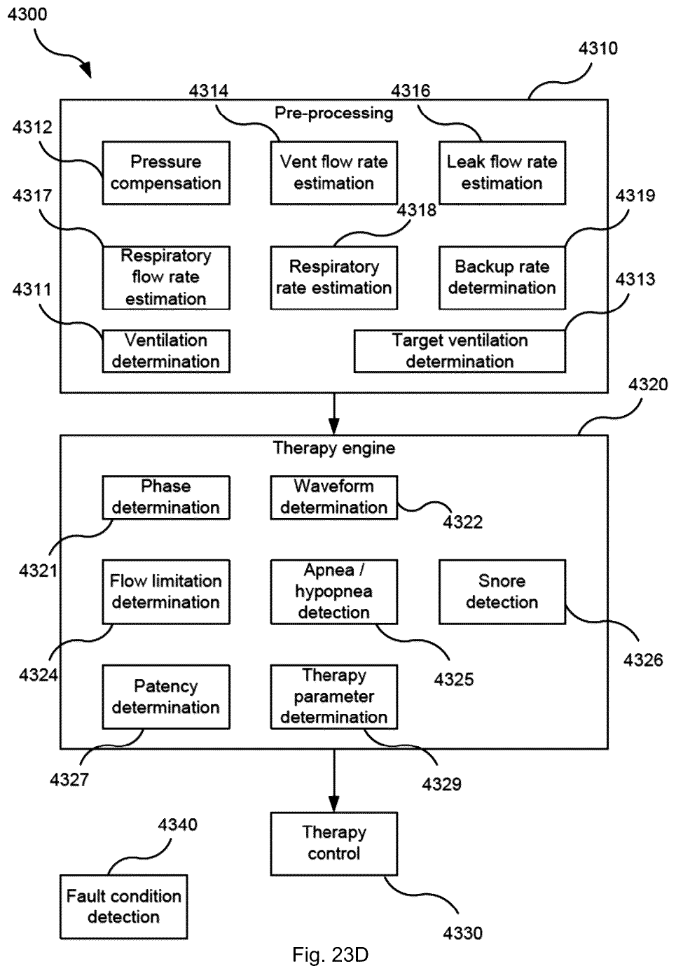

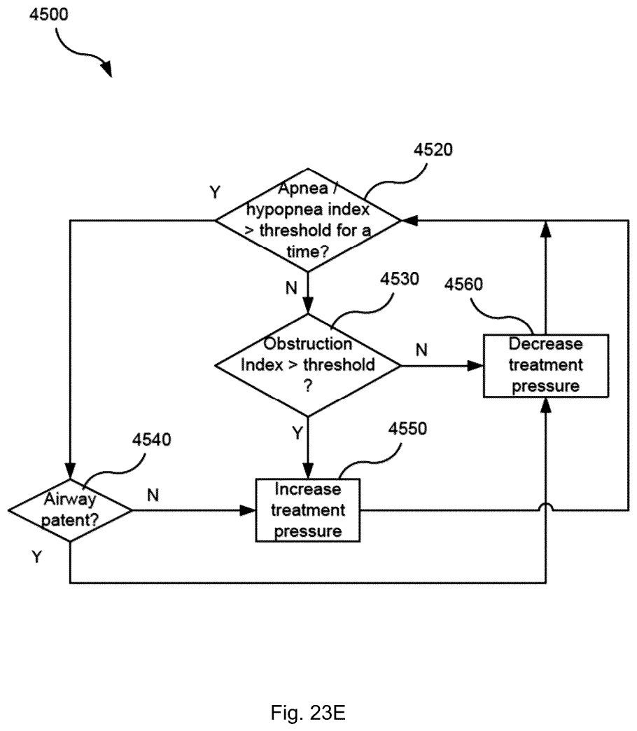

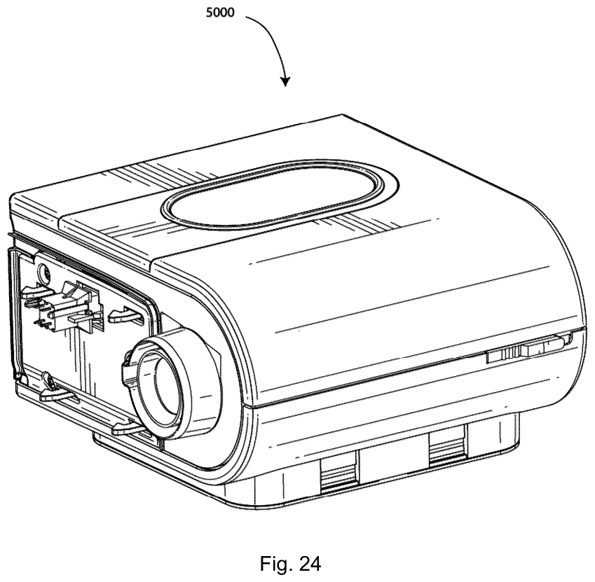

In one form, the such devices may treat and/or monitor a respiratory disorder. One such device may be a respiratory therapy device (RT) such as an RPT device 4000 for supplying a flow of pressurised air to the patient 1000 via an air circuit 4170 leading to a patient interface 3000. The flow of air may be pressure-controlled (for respiratory pressure therapies) or flow-controlled (for flow therapies such as high flow therapy HFT). Thus, RPT devices may also be configured to act as flow therapy devices, such as when using a patient interface that does not use a seal that seals with the patient's respiratory system. In the following description, the RT or RPT device may be considered in reference to FIGS. 21A-24.

### 4.2 Patient Interface

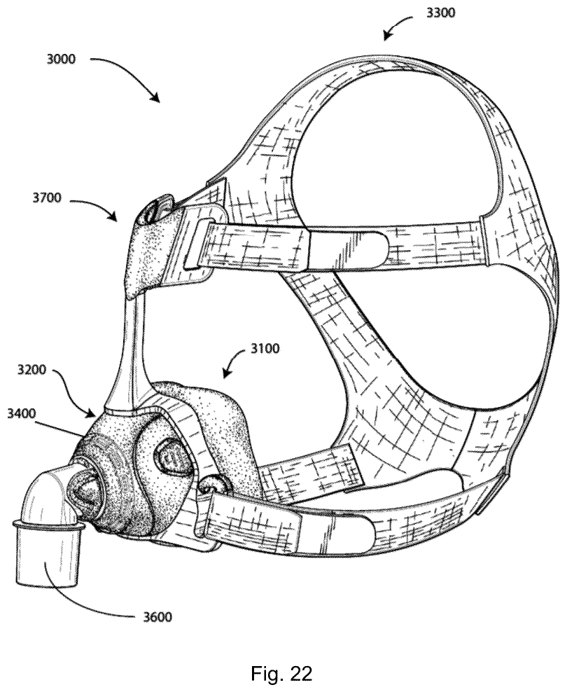

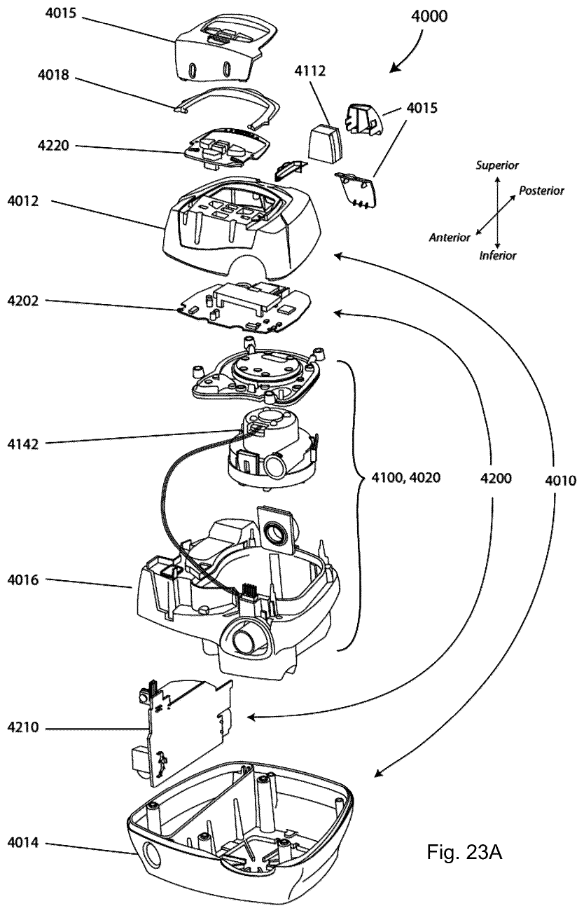

As shown in FIG. 22, a non-invasive patient interface 3000 in accordance with one aspect of the present technology may optionally comprise any of the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to an airway of the patient so as to facilitate the supply of pressurised air to the airway.

### 4.3 RPT Device

An RPT device 4000 in accordance with one example of the present technology may comprise mechanical and pneumatic components 4100, electrical components 4200 and is programmed to execute one or more algorithms 4300. The RPT device 4000 may have an external housing 4010 formed in two parts, an upper portion 4012 and a lower portion 4014. In one form, the external housing 4010 may include one or more panel(s) 4015. The RPT device 4000 may comprise a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying pressurised air (e.g., a blower 4142), an outlet muffler 4124, and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 4.3.1 RPT Device Mechanical & Pneumatic Components

An RPT device 4000 may comprise one or more of the following components in an integral unit. In an alternative form, one or more of the following components may be located as respective separate units.

### 4.3.1.1 Air Filter(s)

An RPT device 4000 in accordance with one form of the present technology may include an air filter 4110, or a plurality of air filters 4110.

In one form, an air inlet filter 4112 is located at the beginning of the pneumatic path upstream of a pressure generator 4140.

In one form, an air outlet filter 4114, for example an antibacterial filter, is located between an outlet of the pneumatic block 4020 and a patient interface 3000.

### 4.3.1.2 Muffler(s)

An RPT device 4000 in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 4.3.1.3 Pressure Generator

In one form of the present technology, a pressure generator 4140 for supplying pressurised air is a controllable blower 4142. For example, the blower 4142 may include a brushless DC motor 4144 with one or more impellers housed in a volute. The pressure generator 4140 may be capable of generating a supply or flow of air, for example at about 120 litres/minute, at a positive pressure in a range from about 4 cmH₂O to about 20 cmH₂O, or in other forms up to about 30 cmH₂O.

The pressure generator 4140 is under the control of the therapy device controller 4240.

In other forms, a pressure generator 4140 may be a piston-driven pump, a pressure regulator connected to a high pressure source (e.g., compressed air reservoir), or a bellows.

### 4.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 are constructed and arranged to generate data representing respective properties of the air flow, such as a flow rate, a pressure or a temperature, at that point in the pneumatic path.

In one form of the present technology, one or more transducers 4270 are located proximate to the patient interface 3000.

In one form, a signal from a transducer 4270 may be filtered, such as by low-pass, high-pass or band-pass filtering.

### 4.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 4.3.1.6 Air Circuit

An air circuit 4170 in accordance with one aspect of the present technology is a conduit or tube constructed and arranged to allow, in use, a flow of air to travel between two components such as the pneumatic block 4020 and the patient interface 3000.

### 4.3.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 4.3.2 RPT Device Electrical Components

### 4.3.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000.

### 4.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 4.3.2.3 Central Controller

In one form of the present technology, the central controller 4230 is a processor suitable to control an RPT device 4000 such as an x86 INTEL processor.

A central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another central controller 4230 suitable to control an RPT device 4000 in accordance with a further alternative form of the present technology includes a member selected from the family ARMS-based 32-bit RISC CPUs. For example, an STR9 series microcontroller from ST MICROELECTRONICS may be used.

In certain alternative forms of the present technology, a 16-bit RISC CPU may be used as the central controller 4230 for the RPT device 4000. For example, a processor from the MSP430 family of microcontrollers, manufactured by TEXAS INSTRUMENTS, may be used.

In another form of the present technology, the central controller 4230 is a dedicated electronic circuit. In another form, the central controller 4230 is an application-specific integrated circuit (ASIC). In another form, the central controller 4230 comprises discrete electronic components.

The central controller 4230 is configured to receive input signal(s) from one or more transducers 4270, one or more input devices 4220, and the humidifier 5000.

The central controller 4230 is configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300, expressed as computer programs stored in a non-transitory computer readable storage medium, such as memory 4260 or other memory described herein. In some forms of the present technology, as previously discussed, the central controller 4230 may be integrated with an RPT device 4000. However, in some forms of the present technology, some methodologies may be performed by a remotely located device or server such as the server previously mentioned. For example, the remotely located device or server may determine control settings for transfer to a ventilator or other RT device such as by detecting respiratory related events and distinguishing them by type by an analysis of stored data such as from any of the sensors described herein.

While the central controller 4230 may comprise a single controller interacting with various sensors 4270, data communications interface 4280, memory 4260, as well as other devices, the functions of controller 4230 may be distributed among more than one controller. Thus, the term “central” as used herein is not meant to limit the architecture to a single controller or processor that controls the other devices. For example, alternative architectures may include a distributed controller architecture involving more than one controller or processor, which may optionally be directly or indirectly in electronic (wired or wireless) communications with the previously described finger sensor or a server in communication with the finger sensor, such as for implementing any of the methodologies described herein. This may include, for example, a separate local (i.e., within RPT device 4000) or remotely located controller that perform some of the algorithms 4300, or even more than one local or remote memory that stores some of the algorithms. In addition, the algorithms when expressed as computer programs may comprise high level human readable code (e.g., C++, Visual Basic, other object oriented languages, etc.) or low/machine level instructions (Assembler, Verilog, etc.). Depending on the functionality of an algorithm(s), such code or instructions may be burnt in the controller, e.g., an ASIC or DSP, or be a run time executable ported to a DSP or general purpose processor that then becomes specifically programmed to perform the tasks required by the algorithm(s).

### 4.3.2.4 Clock

The RPT device 4000 may include a clock 4232 that is connected to the central controller 4230.

### 4.3.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 4.3.2.6 Protection Circuits

An RPT device 4000 in accordance with the present technology may comprise one or more protection circuits 4250.

One form of protection circuit 4250 in accordance with the present technology is an electrical protection circuit.

One form of protection circuit 4250 in accordance with the present technology is a temperature or pressure safety circuit.

### 4.3.2.7 Memory

In accordance with one form of the present technology the RPT device 4000 includes memory 4260, for example non-volatile memory. In some forms, memory 4260 may include battery powered static RAM. In some forms, memory 4260 may include volatile RAM.

Memory 4260 may be located on PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260, such as any of the memories previously described, acts as a non-transitory computer readable storage medium on which is stored computer program instructions expressing the one or more methodologies described herein, such as the one or more algorithms 4300.

### 4.3.2.8 Transducers

Transducers may be internal of the device 4000, or external of the RPT device 4000. External transducers may be located for example on or form part of the air delivery circuit 4170, e.g., at the patient interface 3000. External transducers may be in the form of non-contact sensors such as a Doppler radar movement sensor that transmit or transfer data to the RPT device 4000.

### 4.3.2.8.1 Flow Rate

A flow rate transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In one example, a signal representing total flow rate Qt from the flow transducer 4274 is received by the central controller 4230.

### 4.3.2.8.2 Pressure

A pressure transducer 4272 in accordance with the present technology is located in fluid communication with the pneumatic path. An example of a suitable pressure transducer 4272 is a sensor from the HONEYWELL ASDX series. An alternative suitable pressure transducer is a sensor from the NPA Series from GENERAL ELECTRIC.

In use, a signal from the pressure transducer 4272 is received by the central controller 4230. In one form, the signal from the pressure transducer 4272 is filtered prior to being received by the central controller 4230.

### 4.3.2.8.3 Motor Speed

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 4.3.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol and may optionally communicate with any of the sensors described herein.

In one form, remote external device 4286 is one or more computers, for example a cluster of networked computers and/or server as described herein. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 4.3.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.3.2.10.1 Display Driver

A display driver 4292 receives as an input the characters, symbols, or images intended for display on the display 4294, and converts them to commands that cause the display 4294 to display those characters, symbols, or images.

### 4.3.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 4.3.3 RPT Device Algorithms

### 4.3.3.1 Pre-Processing Module

A pre-processing module 4310 in accordance with the present technology receives, as an input, raw data from a transducer 4270, for example a flow rate sensor 4274 or a pressure sensor 4272, and performs one or more process steps to calculate one or more output values that will be used as an input to another module, for example a therapy engine module 4320.

In one form of the present technology, the output values include the interface or mask pressure Pm, the respiratory flow rate Qr, and the leak flow rate Ql.

In various forms of the present technology, the pre-processing module 4310 comprises one or more of the following algorithms: pressure compensation 4312, vent flow rate estimation 4314, leak flow rate estimation 4316, respiratory flow rate estimation 4317, ventilation determination 4311, target ventilation determination 4313, respiratory rate estimation 4318, and backup rate determination 4319.

### 4.3.3.1.1 Pressure Compensation

In one form of the present technology, a pressure compensation algorithm 4312 receives as an input a signal indicative of the pressure in the pneumatic path proximal to an outlet of the pneumatic block 4020. The pressure compensation algorithm 4312 estimates the pressure drop in the air circuit 4170 and provides as an output an estimated pressure, Pm, in the patient interface 3000.

### 4.3.3.1.2 Vent Flow Rate Estimation

In one form of the present technology, a vent flow rate estimation algorithm 4314 receives as an input an estimated pressure, Pm, in the patient interface 3000 and estimates a vent flow rate of air, Qv, from a vent 3400 in a patient interface 3000.

### 4.3.3.1.3 Leak Flow Rate Estimation

In one form of the present technology, a leak flow rate estimation algorithm 4316 receives as an input a total flow rate Qt and a vent flow rate Qv, and estimates a leak flow rate Ql. In one form, the leak flow rate estimation algorithm 4316 estimates the leak flow rate Ql by calculating an average of the difference between the total flow rate and the vent flow rate Qv over a period sufficiently long to include several breathing cycles, e.g., about 10 seconds.

In one form, the leak flow estimation algorithm 4316 receives as an input a total flow rate Qt, a vent flow rate Qv, and an estimated pressure, Pm, in the patient interface 3000, and estimates a leak flow rate Ql by calculating a leak conductance, and determining a leak flow rate Ql to be a function of leak conductance and the pressure Pm. Leak conductance may be calculated as the quotient of low-pass filtered non-vent flow rate equal to the difference between total flow rate Qt and vent flow rate Qv, and low-pass filtered square root of pressure Pm, where the low-pass filter time constant has a value sufficiently long to include several breathing cycles, e.g., about 10 seconds. The leak flow rate Ql may be estimated as the product of leak conductance and a function of pressure, Pm.

### 4.3.3.1.4 Respiratory Flow Rate Estimation

In one form of the present technology, a respiratory flow rate estimation algorithm 4317 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

In other forms of the present technology, the respiratory flow estimation algorithm 4317 provides a value that acts as a proxy for the respiratory flow rate Qr. Possible proxies for respiratory flow rate include:
Respiratory movement of the chest of the patient 1000
Current drawn by the pressure generator 4140
Motor speed of the pressure generator 4140
Trans-thoracic impedance of the patient 1000

The respiratory flow rate proxy value may be provided by a transducer 4270 in the RPT device 4000, e.g., the motor speed sensor 4276, or a sensor external to the RPT device 4000, such a respiratory movement sensor or a trans-thoracic impedance sensor.

### 4.3.3.1.5 Ventilation Determination

In one form of the present technology, a ventilation determination algorithm 4311 receives an input a respiratory flow rate Qr, and determines a measure Vent indicative of current patient ventilation.

In some implementations, the ventilation determination algorithm 4311 determines a measure of ventilation Vent that is an estimate of actual patient ventilation.

In one such implementation, the measure of ventilation Vent is half the absolute value of respiratory flow, Qr, optionally filtered by low-pass filter such as a second order Bessel low-pass filter with a corner frequency of 0.11 Hz.

In one such implementation, the measure of ventilation Vent is an estimate of gross alveolar ventilation (i.e. non-anatomical-deadspace ventilation). This requires an estimate of anatomical deadspace. One can use the patient's height (or arm-span in cases of severe skeletal deformity) as a good predictor of anatomical deadspace. Gross alveolar ventilation is then equal to a measure of actual patient ventilation, e.g., determined as above, less the product of the estimated anatomical deadspace and the estimated spontaneous respiratory rate Rs.

In other implementations, the ventilation determination algorithm 4311 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar) Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate waveform shape is constant.

In other forms, the ventilation determination algorithm 4311 determines a measure Vent of ventilation that is not based on respiratory flow rate Qr, but is a proxy for the current patient ventilation, such as oxygen saturation (SaO₂), or partial pressure of carbon dioxide (PCO₂), obtained from suitable sensors attached to the patient 1000.

### 4.3.3.1.6 Target Ventilation Determination

In one form of the present technology, a central controller 4230 takes as input the measure of current ventilation, Vent, and executes one or more target ventilation determination algorithms 4313 for the determination of a target value Vtgt for the measure of ventilation.

In some forms of the present technology, there is no target ventilation determination algorithm 4313, and the target ventilation Vtgt is predetermined, for example by hard-coding during configuration of the RPT device 4000 or by manual entry through the input device 4220.

In other forms of the present technology, such as adaptive servo-ventilation (ASV) therapy (described below), the target ventilation determination algorithm 4313 computes the target ventilation Vtgt from a value Vtyp indicative of the typical recent ventilation of the patient 1000.

In some forms of adaptive servo-ventilation therapy, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation therapy, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4313, the recent history is of the order of several minutes, but in any case should be longer than the timescale of Cheyne-Stokes waxing and waning cycles. The target ventilation determination algorithm 4313 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 4.3.3.1.7 Respiratory Rate Estimation

In one form of the present technology, a respiratory rate estimation algorithm 4318 receives as an input a respiratory flow rate, Qr, to the patient 1000, and produces an estimate of the spontaneous respiratory rate Rs of the patient.

The respiratory rate estimation algorithm 4318 may estimate the spontaneous respiratory rate Rs over periods when the patient 1000 is breathing spontaneously, i.e., when the RPT device 4000 is not delivering “backup breaths” (described below). In some forms of the present technology, the respiratory rate estimation algorithm 4318 estimates the respiratory rate over periods when servo-assistance (defined as pressure support minus minimum pressure support) is low, in one implementation less than 4 cmH₂O, as such periods are more likely to reflect spontaneous respiratory effort.

In some forms of the present technology, the respiratory rate estimation algorithm 4318 estimates the respiratory rate over periods of asleep breathing, since the respiratory rate during these periods may be substantially different from the respiratory rate during wake. Anxiety typically results in a higher respiratory rate than that prevailing during sleep. When patients focus on their own breathing process, their respiratory rates are typically lower than those during normal wakefulness or during sleep. Techniques such as described in Patent Application no. PCT/AU2010/000894, published as WO 2011/006199, the entire disclosure of which is hereby incorporated herein by reference, may be used to identify periods of awake breathing from the respiratory flow rate, Qr.

In some forms of the present technology, the respiratory rate estimation algorithm 4318 estimates the spontaneous respiratory rate Rs as the reciprocal of one of a variety of well-known statistical measures of central tendency of breath duration Ttot during the period of interest. In such measures it is desirable to reject, or at least be robust to, outliers. One such measure, trimmed mean, in which the lower and upper K proportions of the sorted breath durations are discarded and the mean calculated on the remaining breath durations, is robust to outliers. For example, when K is 0.25, this amounts to discarding the upper and lower quartiles of breath duration Ttot. The median is another robust measure of central tendency, though this can occasionally give unsatisfactory results when the distribution is strongly bimodal. A simple mean may also be employed as a measure of central tendency, though it is sensitive to outliers. An initial interval filtering stage, in which contiguous time intervals corresponding to implausible respiratory rates (e.g., greater than 45 breaths/minute or less than 6 breaths/minute) are excluded as outliers from the mean calculation, may be employed. Other filtering mechanisms which may be used alone or in combination with interval filtering are to exclude any breaths that are not part of a sequence of N successive spontaneous breaths, where Nis some small integer (e.g., 3), and to exclude the early and late breaths of a sequence of successive spontaneous breaths, e.g., to exclude the first and last breaths of a sequence of four breaths. The rationale for the latter mechanism is that the first and the last breaths in particular, and the early and late breaths in general, of a sequence of spontaneous breaths may be atypical; for example, the first spontaneous breath may occur as a result of an arousal, and the last spontaneous breath may be longer because of the decreasing respiratory drive which results in the backup breath which ends the sequence of spontaneous breaths.

In some forms of the present technology, the respiratory rate estimation algorithm 4318 makes an initial estimate of the spontaneous respiratory rate Rs using an initial period of estimation, to enable the subsequent processing in the therapy engine module 4320 to begin, and then continuously updates the estimate of the spontaneous respiratory rate Rs using a period of estimation that is longer than the initial period of estimation, to improve statistical robustness. For example, the initial period of estimation may be 20 minutes of suitable spontaneous breaths, but the period of estimation may then progressively increase up to some maximum duration, for example 8 hours. Rather than a rolling window of this duration being used for this estimation, low-pass filters on breath duration may be used, with progressively longer response times (more precisely, progressively lower corner frequencies) as the session proceeds.

In some forms, a suitably processed short-term (e.g., 10-minute) measure of central tendency, such as trimmed mean, may be input to a suitable low-pass filter to give an estimate Rs which changes on the time scale of hours or longer. This has the advantage that potentially large amounts of breath duration data do not need to be stored and processed, as might occur if a trimmed mean needs to be calculated on a moving window of breath duration data lasting hours or days.

In some forms of the present technology, respiratory rates measured over short periods of time, and in particular over one breath, may also be used instead of breath duration in the above-described measures of central tendency, giving generally similar but not identical results.

### 4.3.3.2 Therapy Engine Module

In one form of the present technology, a therapy engine module 4320 receives as inputs one or more of a pressure, Pm, in a patient interface 3000, a respiratory flow rate of air to a patient, Qr, and an estimate Rs of the spontaneous respiratory rate, and provides as an output one or more therapy parameters. In various forms, the therapy engine module 4320 comprises one or more of the following algorithms: phase determination 4321, waveform determination 4322, inspiratory flow limitation determination 4324, apnea/hypopnea determination 4325, snore detection 4326, airway patency determination 4327, and therapy parameter determination 4329.

### 4.3.3.2.2 Waveform Determination

In one form of the present technology, the therapy control module 4330 controls a pressure generator 4140 to provide a treatment pressure Pt that varies as a function of phase of a breathing cycle of a patient according to a waveform template.

### 4.3.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of gas whose mask pressure Pm at the patient interface 3000 is equal to the treatment pressure Pt.

### 4.5 Glossary

For the purposes of the present disclosure, in certain forms of the present technology, one or more of the following definitions may apply. In other forms of the present technology, alternative definitions may apply.

### 4.5.1 General

Air: In certain forms of the present technology, air may be taken to mean atmospheric air, and in other forms of the present technology air may be taken to mean some other combination of breathable gases, e.g., atmospheric air enriched with oxygen.

Respiratory Pressure Therapy (RPT): The delivery of a supply of air to the airways at a treatment pressure that is typically positive with respect to atmosphere.

Continuous Positive Airway Pressure (CPAP) therapy: Respiratory pressure therapy in which the treatment pressure is approximately constant through a breathing cycle of a patient. In some forms, the pressure at the entrance to the airways will be slightly higher during exhalation, and slightly lower during inhalation. In some forms, the pressure will vary between different breathing cycles of the patient, for example, being increased in response to detection of indications of partial upper airway obstruction, and decreased in the absence of indications of partial upper airway obstruction.

Patient: A person, whether or not they are suffering from a respiratory disease.

Automatic Positive Airway Pressure (APAP) therapy: CPAP therapy in which the treatment pressure is automatically adjustable, e.g., from breath to breath, between minimum and maximum limits, depending on the presence or absence of indications of SDB events.

### 4.5.2 Aspects of the Breathing Cycle

Apnea: According to some definitions, an apnea is said to have occurred when respiratory flow rate falls below a predetermined threshold for a duration, e.g., 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate, or respiratory rate (Rs): The rate of spontaneous respiration of a patient, usually measured in breaths per minute.

Duty cycle: The ratio of inhalation time, Ti to total breath duration, Ttot.

Effort (breathing): The work done by a spontaneously breathing person attempting to breathe.

Expiratory portion of a breathing cycle: The period from the start of expiratory flow to the start of inspiratory flow.

Flow limitation: The state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: A reduction in flow, but not a cessation of flow. In one form, a hypopnea may be said to have occurred when there is a reduction in flow below a threshold for a duration. In one form in adults, the following either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow rate (Qpeak): The maximum value of flow during the inspiratory portion of the respiratory flow rate waveform.

Respiratory flow/airflow rate, patient flow/airflow rate (Qr): These synonymous terms may be understood to refer to the RPT device's estimate of respiratory airflow rate, as opposed to “true respiratory flow rate” or “true respiratory airflow rate”, which is the actual respiratory flow rate experienced by the patient, usually expressed in litres per minute.

Tidal volume (Vt): The volume of air inhaled or exhaled during normal breathing, when extra effort is not applied.

Inhalation Time (Ti): The duration of the inspiratory portion of the respiratory flow rate waveform.

Exhalation Time (Te): The duration of the expiratory portion of the respiratory flow rate waveform.

(total) Time, or breath duration (Ttot): The total duration between the start of the inspiratory portion of one respiratory flow rate waveform and the start of the inspiratory portion of the following respiratory flow rate waveform.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.5.3 RPT Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow rate will be given the symbol Q. ‘Flow rate’ is sometimes shortened to simply ‘flow’. Total flow rate, Qt, is the flow of air leaving the RPT device. Vent flow rate, Qv, is the flow of air leaving a vent to allow washout of exhaled gases. Leak flow rate, Ql, is the flow rate of unintentional leak from a patient interface system. Respiratory flow rate, Qr, is the flow of air that is received into the patient's respiratory system.

Leak: The word leak will be taken to be an unintended flow of air. In one example, leak may occur as the result of an incomplete seal between a mask and a patient's face. In another example leak may occur in a swivel elbow to the ambient.

Pressure: Force per unit area. Pressure may be measured in a range of units, including cmH₂O, g-f/cm², hectopascal. 1 cmH₂O is equal to 1 g-f/cm² and is approximately 0.98 hectopascal. In this specification, unless otherwise stated, pressure is given in units of cmH₂O. The pressure in the patient interface (mask pressure) is given the symbol Pm, while the treatment pressure, which represents a target value to be achieved by the mask pressure Pm at the current instant of time, is given the symbol Pt.

### 4.5.4 Terms for Ventilators

Adaptive Servo-Ventilator (ASV): A servo-ventilator that has a changeable rather than a fixed target ventilation. The changeable target ventilation may be learned from some characteristic of the patient, for example, a respiratory characteristic of the patient.

Backup rate: A parameter of a ventilator that establishes the respiratory rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

End expiratory pressure (EEP): Desired mask pressure which the ventilator will attempt to achieve at the end of the expiratory portion of the breath.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Servo-assistance: Pressure support minus minimum pressure support.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the inspiratory portion of the breathing cycle by the patient's efforts.

Typical recent ventilation: The typical recent ventilation Vtyp is the value around which recent measures of ventilation over some predetermined timescale tend to cluster, that is, a measure of the central tendency of the measures of ventilation over recent history.

Ventilator: A mechanical device that provides pressure support to a patient to perform some or all of the work of breathing.

### FURTHER REMARKS

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

Although the present invention has been illustrated by reference to specific embodiments, it will be apparent to those skilled in the art that the invention is not limited to the details of the foregoing illustrative embodiments, and that the present technology may be embodied with various changes and modifications without departing from the scope thereof. The present examples are therefore to be considered in all respects as illustrative and not restrictive, the scope of the technology being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the technology are capable of operating according to the present technology in other sequences, or in orientations different from the one(s) described or illustrated above.

1. A method of one or more servers for transferring access to patient data from a first organization to a second organization, the patient data comprising therapy data for one or more patients, the method comprising:
generating, by the one or more servers, a user interface;
receiving job input data via the user interface, the job input data comprising transfer information indicating the first organization and the second organization;
generating, by the one or more servers, a transfer job based on the job input data, the transfer job comprising list data indicative of the one or more patients, the one or more patients being in association with the first organization;
executing, by the one or more servers, the generated transfer job, the executing comprising iterating through the list data and, for each patient indicated by the list data, communicating transfer commands to a plurality of data services of the one or more servers, whereby access to the patient data, of a central database, associated with the one or more patients indicated by the list data transfers from the first organization to the second organization; and
generating, for a display on the user interface, transfer job status information concerning the status of the executing.

2. The method of claim 1, wherein the plurality of data services comprises a billing rules system, a compliance rules system, a list service and a device type subset data system.

3. The method of any one of claims 1 to 2, wherein the user interface prompts for input of a target entity identification and source entity identification and generates a network communication to a merge coordinator service with the input target entity identification and input source entity identification.

4. The method of claim 3, wherein the merge coordinator service receives the network communication with the entity identification and source entity identification and queries the central database to generate a list of patients associated with the source entity identification.

5. The method of any one of claims 3 to 4, wherein the user interface further prompts for input of a clinical user or location of the source entity and generates the network communication to the merge coordinator service with the clinical user or location of the source entity.

6. The method of any one of claims 3 to 5, wherein the user interface further prompts for input of a clinical user or location of the target entity and generates the network communication to the merge coordinator service with the clinical user or location of the target entity.

7. The method of any one of claims 3 to 6, wherein the user interface further prompts for input of a clinical user or location of the target entity and generates the network communication to the merge coordinator service with the clinical user or location of the target entity.

8. The method of any one of claims 4 to 7, wherein the merge coordinator service iteratively communicates with a patient transfer service for each patient of the list of patients.

9. The method of claim 8 wherein the patient transfer service communicates with the plurality of data services and the central database in response to each iterative communication from the merge coordinator service to transfer access to a patient of the list of patients from the first organization to the second organization.

10. The method of any one of claims 1 to 9 wherein the user interface presents a restart icon in association with a failed transfer, and wherein the user interface is configured to communicate a restart request for the failed transfer in response to activation of the restart icon.

11. The method of claims 3 and 10, wherein the merge coordinator service communicates with a database to store the transfer job status information.

12. The method of any one of claims 1 to 11, wherein the patient data for which access is transferred by the executing comprises therapy device data, and wherein the method further comprises communicating, with one or more servers, one or more control commands to a patient therapy device based on the therapy device data for making a change to an operation of the therapy device.

13. A system for transferring access to patient data from a first organization to a second organization, the patient data comprising therapy data for one or more patients, the system comprising:
one or more processors; and
one or more computer-readable storage media having program instructions thereon, which when executed by the one or more processors cause the system to:
generate a user interface;
receive job input data via the user interface, the job input data comprising transfer information indicating the first organization and the second organization;
generate a transfer job based on the job input data, the transfer job comprising list data indicative of the one or more patients, the one or more patients being in association with the first organization;
execute the generated transfer job, the execution comprising iterating through the list data and, for each patient indicated by the list data, communicating transfer commands to a plurality of data services of the one or more servers, whereby access to the patient data, of a central database, associated with the one or more patients indicated by the list data transfers from the first organization to the second organization; and
generate, for a display on the user interface, transfer job status information concerning the status of the execution.

14. The system of claim 13, wherein the plurality of data services comprises a billing rules system, a compliance rules system, a list service and a device type subset data system.

15. The system of any one of claims 13 to 14, wherein the user interface is configured to prompt for input of a target entity identification and source entity identification and generates a network communication to a merge coordinator service with the input target entity identification and input source entity identification.

16. The system of claim 15, wherein the merge coordinator service is configured to receive the network communication with the entity identification and source entity identification and is further configured to query the central database to generate a list of patients associated with the source entity identification.

17. The system of any one of claims 15 to 16, wherein the user interface is configured to further prompt for input of a clinical user or location of the source entity and is further configured to generate the network communication to the merge coordinator service with the clinical user or location of the source entity.

18. The system of any one of claims 15 to 16, wherein the user interface is configured to further prompt for input of a clinical user or location of the target entity and is configured to generate the network communication to the merge coordinator service with the clinical user or location of the target entity.

19. The system of any one of claims 15 to 18, wherein the user interface is further configured to prompt for input of a clinical user or location of the target entity and is configured to generate the network communication to the merge coordinator service with the clinical user or location of the target entity.

20. The system of any one of claims 16 to 19, wherein the merge coordinator service is configured to iteratively communicate with a patient transfer service for each patient of the list of patients.

21. The system of claim 20 wherein the patient transfer service is configured to communicate with the plurality of data services and the central database in response to each iterative communication from the merge coordinator service to transfer access to a patient of the list of patients from the first organization to the second organization.

22. The system of any one of claims 13 to 21 wherein the user interface is configured to present a restart icon in association with a failed transfer, and wherein the user interface is configured to communicate a restart request for the failed transfer in response to activation of the restart icon.

23. The system of any one of claims 13 to 22, wherein the patient data for which access is transferred by the execution of the generated transfer job comprises therapy device data, and wherein the system is further figured to communicate, by one or more servers, one or more control commands to a patient therapy device based on the therapy device data for making a change to an operation of the therapy device.

24. A method of one or more servers for transferring access to patient data from a first organization to a second organization, the patient data comprising therapy data for a patient using a therapy device, the method comprising:
generating, by the one or more servers, a user interface for transferring access to the patient data;
receiving input data from the second organization, with the user interface, the input data comprising device identification indicia of the therapy device;
based on receiving the device identification indicia, communicating transfer commands to a plurality of services of the one or more servers, whereby access to the patient data for the patient of a central database transfers from the first organization to the second organization; and
generating, for a display on the user interface, an indication of the transfer of access.

25. The method of claim 24, wherein the user interface prompts for input of serial number of the therapy device, and activates a search of the central database with the serial number.

26. The method of claim 25, wherein in response to search, the user interface displays patient data for a patient associated with: (a) the first organization and (b) the device identification indicia of the therapy device.

27. The method of any one of claims 24 to 26, wherein the user interface prompts for entry of confirmation of authority to request transfer of the patient from the first organization to the second organization.

28. The method of any one of claims 24 to 27, wherein the user interface prompts for entry of a clinical user of the second organization and/or a location of the second location.

29. The method of any one of claims 24 to 27, wherein the user interface displays the indication of the transfer of access to the second organization.

30. The method of any one of claims 24 to 27, further comprising generating a further user interface and communicating the further user interface to the first organization, the further user interface comprising a display of the indication of the transfer of access.

31. The method of any one of claims 24 to 30, wherein the patient data, for which access is transferred based on the communicated transfer commands, comprises therapy device data associated with the therapy device, and wherein the method further comprises communicating, with one or more servers, one or more control commands to the therapy device based on the therapy device data for making a change to an operation of the therapy device.

32. A system transferring access to patient data from a first organization to a second organization, the patient data comprising therapy data for one or more patients, the system comprising:
one or more processors; and
one or more computer-readable storage media having program instructions thereon, which when executed by the one or more processors cause the system to:
generate a user interface for transferring access to the patient data;
receive input data from the second organization, with the user interface, the input data comprising device identification indicia of the therapy device;
based on the received device identification indicia, communicate transfer commands to a plurality of services of the one or more servers, whereby access to the patient data for the patient of a central database transfers from the first organization to the second organization; and
generate, for a display on the user interface, an indication of the transfer of access.

33. The system of claim 32, wherein the user interface is configured to prompt for input of serial number of the therapy device, and activate a search of the central database with the serial number.

34. The system of claim 33, wherein in response to the search, the user interface is configured to display patient data for a patient associated with: (a) the first organization and (b) the device identification indicia of the therapy device.

35. The system of any one of claims 32 to 34, wherein the user interface is configured to prompt for entry of confirmation of authority to request transfer of the patient from the first organization to the second organization.

36. The system of any one of claims 32 to 35, wherein the user interface is configured to prompt for entry of a clinical user of the second organization and/or a location of the second location.

37. The system of any one of claims 32 to 36, wherein the user interface is configured to display the indication of the transfer of access to the second organization.

38. The system of any one of claims 32 to 37, wherein the system is further configured to generate a further user interface and communicate the further user interface to the first organization, wherein the further user interface is configured to display the indication of the transfer of access.

39. The system of any one of claims 32 to 37, wherein the patient data for which access is transferred based on the communicated transfer commands, comprises therapy device data associated with the therapy device, and wherein the system is further configured to communicate, with one or more servers, one or more control commands to the therapy device based on the therapy device data for making a change to an operation of the therapy device.
